(19) United States
(12) Patent Application Publication
(10) Pub. No.: US 2009/0220562 A1
(43) Pub. Date: Sep. 3, 2009

(54) Osteogenic and anti-adipogenic oxysterols

(76) Inventor: Farhad Parhami, Los Angeles, CA (US)

Correspondence Address: VENABLE LLP, P.O. BOX 34385, WASHINGTON, DC, 20043-9998, US

(21) Appl. No.: 11/991,322

(22) PCT Filed: Sep. 5, 2006
(86) PCT No.: PCT/US2006/034374

§ 371 Date: May 11, 2009

Related U.S. Application Data
(60) Provisional application No. 60/714,063, filed on Sep. 2, 2005.

(51) Int. Cl. A61K 9/00; A61K 31/56; A61K 38/28; A61K 38/22; A61K 33/16; A61P 19/00

(52) U.S. Cl. 424/423; 514/182; 514/3; 514/12; 424/676

(57) ABSTRACT

The present invention discloses osteogenic and anti-adipogenic oxysterols. Agents and methods for protecting, blocking or rescuing marrow stromal cells from the inhibitory effects of oxidative stress on their osteoblastic cellular differentiation are disclosed. Exemplary agents include oxysterols alone or in synergistic combinations, as well as hedgehog or Wnt signaling activators. The synergistic effects of oxysterols and bone morphogenic proteins are also disclosed.

This research is sponsored by National Institutes of Health/National Institutes on Aging Pepper Center, Grant No. IP60 AG 10415-11. The Government has certain rights in this invention.

### BACKGROUND OF THE INVENTION

Normal bone remodeling, which occurs throughout the adult life in order to preserve the integrity of the skeleton, involves bone resorption by osteoclasts and bone formation by osteoblasts. Thus, any interference between the balance in bone formation and bone resorption can affect bone homeostasis, bone formation and repair.

The osteoblasts come from a pool of marrow stromal cells (also known as mesenchymal stem cells; MSC). These cells. are present in a variety of tissues and are prevalent in bone marrow stroma. MSC are pluripotent and can differentiate into osteoblasts, chondrocytes, fibroblasts, myocytes, and adipocytes.

Osteoporosis is a major cause of morbidity and mortality in the elderly and the annual cost to the U.S. health care system is at least ten billion dollars. Both men and women suffer from osteoporotic bone loss with age. Decreases in sex hormones with age are thought to impact these detrimental changes. For example, osteoporosis increases in women after menopause.

Accumulating evidence suggests that the number and activity of osteoblastic cells decrease with age, however the reason for this change is not clear. Additionally, there is an increase in formation of adipocytes in osteoporotic bone marrow that appears to be at the expense of osteoblast formation. Moreover, the volume of adipose tissue in bone increases with age in normal subjects, and is substantially elevated in age-related osteoporosis, with the number of adipocytes adjacent to bone trabeculae increasing in parallel to the degree of trabecular bone loss. Based on this and similar observations, it has been suggested that bone loss in age-related osteoporosis is at least in part due to a shift from osteoblastic differentiation to the adipocytic pathway.

Bone fracture healing is impaired in the elderly, and others demonstrating a reduced number and activity of the MSC that would normally migrate into the fracture site and allow for new bone formation to occur.

At present, the only treatments for osteoporosis are those that target bone resorption by osteoclasts. These FDA approved therapeutics include the bisphosphonates, hormone replacement therapies, such as selective estrogen receptor modulators, calcitonin, and vitamin D/calcium supplementation. However, these treatments result in only small improvements in bone mass, and are not sufficient for total prevention or treatment of osteoporosis.

Currently, the only FDA approved anabolic agent for the treatment of osteoporosis is parathyroid hormone (PTH). PTH is currently thought to increase bone formation by inhibiting osteoblast apoptosis. PTH has been found to increase bone mass upon intermittent injection and reduce bone fracture incidence in osteoporotic patients. However, the dose must be strictly regulated since continuous treatment with PTH and/or its accumulation may have adverse systemic effects upon the patient. Additionally, PTH treatment is quite expensive. Consequently, PTH treatment has been reserved for only the most severely osteoporotic patients.

Other potential therapeutics for enhancing bone formation by osteoblasts include sodium fluoride and growth factors that have a positive effect on bone (for example insulin-like growth factors I and II and transforming growth factor beta). However, thus far these factors have had undesirable side effects.

The use of stem cells for treating bone related disorders in humans has also been examined. For example, osteogenesis imperfecta is a skeletal disease in which the patient's osteoblasts do not make collagen I in a proper form, resulting in the brittle bones. Infusion of osteoblastic progenitor stem cells from a healthy individual into a diseased individual has been shown to improve bone density in these patients.

Therefore, agents and methods for regulating bone homeostasis, bone formation and bone repair are desired.

Osteoporotic bone loss may result in increased fracture incidence at the hip, spine, and other sites. (Cummings and Melton 2002. Epidemiology and outcomes of osteoporotic fractures. The Lancet 359:1761-1767; and Ettinger 2003. Aging bone and osteoporosis. Arch Intern Med 163:2237-2246.) As discussed, osteoporosis is associated with a marked decrease in osteoblast number and bone forming activity (Quarto, et al. 1995. Bone progenitor cell deficits and the age-associated decline in bone repair capacity. Calcif Tissue Int 56:123-129; Mullender et al. 1996. Osteocyte density-changes in aging and osteoporosis. Bone 18:109-113; Chan and Duque 2002. Age-related bone loss: old bone, new facts. Gerontology 48:62-71; Ichioka et al. 2002. Prevention of senile osteoporosis in SAMP6 mice by intrabone marrow injection of allogeneic bone marrow cells. Stem Cells 20:542-551; and Chen et al. 2002. Age-related osteoporosis in biglycan-deficient mice is related to defects in bone marrow stromal cells. J Bone Miner Res 17:331-340.) Strategies for increasing bone formation by osteoblasts may be developed to improve skeletal health and prevent osteoporotic bone loss (Mundy 2002. Directions of drug discovery in osteoporosis. Annu Rev Med 53:337-354; and Rodan and Martin 2002. Therapeutic approaches to bone diseases. Science 289:1508-1514).

Although the reason(s) for the decrease in osteoblastic activity and bone formation with age and after menopause is not clearly understood, increased oxidative stress on bone cells may in part explain the reason for this decrease in osteogenic activity. Both aging and menopause are associated with increased oxidative stress and decreased antioxidant defense mechanisms (Sohal et al. 2002. Mechanisms of aging: an appraisal of the oxidative stress hypothesis. Free Radical Biol Med 33:575-586; and Chang et al. 2002. Effects of hormonal replacement therapy on oxidative stress and total antioxidant capacity in postmenopausal hemodialysis patients. Ren Fail 24:49-57). Increased levels of urinary isoprostane, 8-iso-PGF_{2α}, (a biomarker of oxidative stress), is negatively associated with bone mineral density in humans (Basu et al. 2001. Association between oxidative stress and bone mineral density. Biochem Biophys Res Commun 288:275-279. 12). Furthermore, a marked decrease in plasma antioxidants including vitamins C and E, superoxide dismutase, and glutathione peroxidase was reported in aged osteoporotic women compared to controls (Maggio et al. 2003. Marked decrease in plasma antioxidants in aged osteoporotic women: results of a cross-sectional study. Clin Endocrinol & Metab 88:1523-1527). In addition, some epidemiological studies have demonstrated the protective effects of increased dietary antioxidants on bone health (Melhus et al. 1999. Smoking, antioxidant vitamins, and the risk of hip fracture. J Bone Miner Res 14:129-135; and Schaafsma et al. 2001. Delay of natural bone loss by higher intake of specific minerals and vitamins. Crit Rev Food Sci Nutr 41:225-249).

Oxidative stress may negatively impact bone homeostasis by stimulating osteoclastogenesis and bone resorption (Garrett et al. 1990. Oxygen-derived free radicals stimulate osteoclastic bone resorption in rodent bone in vitro and in vivo. J Clin Invest 85:632-639), and by inhibiting osteoblastic differentiation of osteoprogenitor cells (Mody et al. 2001. Differential effects of oxidative stress on osteoblastic differentiation of vascular and bone cells. Free Radical Res & Med 31:509-519). Oxidative stress induced by xanthine/xanthine oxidase or by minimally oxidized LDL (MM-LDL) inhibits osteoblastic differentiation and mineralization in cultures of M2-10B4 (M2) pluripotent marrow stromal cells that can differentiate into osteoblastic cells, and in cultures of MC3T3-E1 calvarial preosteoblasts. Id.

Therefore methods and compositions to protect, block or rescue osteogenic cells from the negative effects of oxidative stress may be clinically useful to induce osteogenesis and to combat osteoporotic bone loss.

### SUMMARY OF THE INVENTION

The present invention is related to agents and methods for maintaining bone homeostasis, enhancing bone formation and/or enhancing bone repair.

The invention includes a method of inducing osteoblastic differentiation of mammalian mesenchymal stem cells including treating mammalian mesenchymal cells with at least one oxysterol, wherein the at least one oxysterol is selected from the group comprising 5-cholesten-3beta, 20alpha-diol 3-acetate (also known as 20A-hydroxycholesterol), 24(S)-hydroxycholesterol (also known as cerebrosterol), 24(S),25-epoxycholesterol, and 26-hydroxycholesterol, 4 beta-hydroxychloesterol, or an active portion of any one of 5-cholesten-3beta, 20alpha-diol 3-acetate, 24(S)-hydroxycholesterol, 24(S),25-epoxycholesterol, 26-hydroxycholesterol or 4 beta-hydroxychloesterol). These molecules share structural similarities with other molecules which have osteogenic properties which may also be used to induce osteoblastic differentiation, 22(R)-hydroxycholesterol, 22(S)-hydroxycholesterol, 20(S)-hydroxycholesterol. Active portions of these oxysterols may also be useful in this invention.

The invention includes a method of inducing osteoblastic differentiation of mammalian mesenchymal stem cells including treating mammalian mesenchymal cells with at least one agent, wherein the agent is selected from the group of oxysterols and portions of oxysterols identified above.

The invention includes a method of stimulating mammalian cells to express a level of a biological marker of osteoblastic differentiation which is greater than the level of a biological marker in untreated cells,. comprising exposing a mammalian cell to a selected dose of at least one agent, wherein the at least one agent is selected from the group of oxysterols and portions of oxysterols identified above.

The invention includes a method of inhibiting osteoblastic differentiation in. mammalian mesenchymal cells by oxysterols comprising treating mammalian mesenchymal cells with a hedgehog signaling inhibitor, and a method of inducing osteoblastic differentiation in mammalian mesenchymal cells comprising treating mammalian mesenchymal cells with a hedgehog signaling activator.

The invention includes a method of effecting the cellular hedgehog signaling pathway by using at least one oxysterol or an active portion of an oxysterol molecule to cause a biological effect regulated by the hedgehog signaling pathway, including contacting cells with at least one oxysterol or an active portion of an oxysterol; and observing the cells for an indicator of the desired biological effect.

The invention includes a method of inhibiting osteoblastic differentiation by oxysterols in mammalian mesenchymal cells comprising treating mammalian mesenchymal cells with a Wnt signaling inhibitor, and a method of inducing osteoblastic differentiation in mammalian cells by oxysterols comprising treating mammalian mesenchymal cells with a Wnt signaling activator.

The invention also includes a method of inhibiting adipocyte differentiation of mammalian mesenchymal stem cells including treating mammalian mesenchymal cells with at least one agent, wherein the at least one agent is selected from the group of oxysterols and portions of oxysterols identified above.

The invention includes a method of treating a patient exhibiting clinical symptoms of osteoporosis comprising administering at least one agent at a therapeutically effective dose in an effective dosage form at a selected interval to ameliorate the symptoms of the osteoporosis, wherein the at least one oxysterol is selected from the group of oxysterols and portions of oxysterols identified above.

The invention includes a method of treating a patient to induce bone formation including: 1) harvesting mammalian mesenchymal stem cells; 2) treating the mammalian mesenchymal cells with at least one agent, wherein the at least on agent induces the mesenchymal stem cells to express at least one cellular marker of osteoblastic differentiation; and 3) administering the differentiated cells to the patient, wherein the at least one agent is selected from the group of oxysterols and portions of oxysterols identified above.

The invention includes an implant for use in the human body comprising, a substrate having a surface, wherein at least the surface of the implant includes at least one agent selected from the group of oxysterols and portions of oxysterols identified above in an amount sufficient to induce bone formation in the surrounding bone tissue.

The invention further includes a medicament for use in the treatment of bone disorders comprising a therapeutically effective dosage of at least one oxysterol selected from the group of oxysterols and portions of oxysterols identified above.

The invention includes method of inducing osteoblastic differentiation of mammalian mesenchymal stem cells including treating mammalian mesenchymal cells with at least one oxysterol and at least one bone morphogenic protein, wherein the at least one oxysterol is selected from the group of oxysterols and portions of oxysterols identified above.

The invention includes a method of stimulating mammalian cells to express a level of a biological marker of osteoblastic differentiation which is greater than the level of a biological marker in untreated cells, comprising exposing a mammalian cell to a selected dose of at least one oxysterol and at least one bone morphogenic protein, wherein the at least one oxysterol is selected from the group of oxysterols and portions of oxysterols identified above, and wherein the at least one bone morphogenic protein is selected from the group comprising BMP2, BMP 7, or BMP 14.

The invention includes a method of treating a patient to increase the differentiation of marrow stromal cells into osteoblasts comprising administering at least one oxysterol and at least one bone morphogenic protein at a therapeutically effective dose in an effective dosage form at a selected interval to increase the number of osteoblasts present in bone tissue, wherein the at least one oxysterol is selected from the group of oxysterols and portions of oxysterols identified above, wherein the at least one bone morphogenic protein is selected from the group comprising BMP2, BMP 7, or BMP 14.

The invention includes a method of treating a patient to induce bone formation comprising administering at least one oxysterol and at least one bone morphogenic protein at a therapeutically effective dose in an effective dosage form at a selected interval to increase bone mass and enhance bone repair, wherein the at least one oxysterol is selected from the group of oxysterols and portions of oxysterols identified above, wherein the at least one bone morphogenic protein is selected from the group comprising BMP2, BMP 7, or BMP 14.

The invention includes a method of blocking the inhibition of osteoblastic differentiation of mammalian mesenchymal stem cells under conditions of oxidative stress including concurrently treating mammalian mesenchymal cells with at least one oxysterol selected from the group of oxysterols and portions of oxysterols identified above.

The invention includes a method of protecting from inhibition of osteoblastic differentiation of mammalian mesenchymal stem cells under conditions of oxidative stress including pre-treating mammalian mesenchymal cells with at least one oxysterol selected from the group of oxysterols and portions of oxysterols identified above.

The invention includes a method of inhibiting adipocyte differentiation of mammalian mesenchymal stem cells including treating mammalian mesenchymal cells with at least one oxysterol, wherein the at least one oxysterol is selected from the group of oxysterols and portions of oxysterols identified above.

Agents and Methods for Osteogenic Oxysterols Inhibition of Oxidative Stress on Osteogenic Cellular Differentiation. The invention may further include any portion of the oxysterol molecule which is found to be active in effecting osteoblastic differentiation or bone formation. The invention may further include the activation of a molecule at which the oxysterols are active in effecting osteoblastic differentiation or bone formation. The invention may also include other lipid molecules or analogs designed to mimic the active portions of the above oxysterols, which would act similarly to the parent molecules, via similar mechanisms of action, and/or via similar receptors that would have a positive impact osteoblastic differentiation or bone formation.

The invention may also include the use of agents which induce osteoblastic bone formation. Agents which may be useful in this invention include, but are not limited to bone morphogenic proteins (BMPs), PTH, sodium fluoride and growth factors, such as insulin-like growth factors I and II and transforming growth factor beta. The invention may include the use of agents which inhibit osteoclastic bone resorption. Agents which may be useful in this invention to effect osteoclastic bone resorption include, but are not limited to, bisphosphonates, the selective estrogen receptor modulators, calcitonin, and vitamin D/calcium supplementation.

The invention may include a method of systemic delivery or localized treatment with agents for maintaining bone homeostasis, enhancing bone formation and/or enhancing bone repair. The invention may include a method of systemic delivery or localized treatment with differentiated osteoblastic cells for maintaining bone homeostasis, enhancing bone formation and/or enhancing bone repair.

The invention may also include implants having coatings of substances or seeded with differentiated cells for inducing bone homeostasis, formation or enhancing bone repair. The invention may also include the application of substances or differentiated cells at a site where bone formation or bone repair is desired.

### BRIEF DESCRIPTION OF THE DRAWINGS

FIG. 1 depicts a flowchart of one method according to this invention.

FIG. 2 depicts two embodiments of the present invention.

FIG. 3: A) is a bar graph depicting the effect of various oxysterols on alkaline phosphatase activity in M2 cells; B) is a bar graph depicting the effect of a combination of oxysterols at various doses on alkaline phosphatase activity in M2 cells; C) is a depiction of von Kossa staining of M2 cells exposed to various conditions; D) is a bar graph depicting the effect of a combination of oxysterols at various doses on calcium incorporation in M2 cells; E) is a radiogram of Northern blotting for osteocalcin mRNA in M2 cells exposed to a control or combination of oxysterols for 4 or 8 days; F) is a bar graph depicting the relative densometric units of osteocalcin mRNA in M2 cells exposed to a control or combination of oxysterols for 4 or 8 days.

FIG. 4: A) is a bar graph depicting the effect of various oxysterols at various doses on M2 cells; B) is a bar graph depicting the effect of various oxysterols at various doses on M2 cells; C) is a bar graph depicting the effect of duration of treatment with oxysterols on M2 cells; D) is a bar graph depicting the effect of various dose combinations of oxysterols on M2 cells; E) is a bar graph depicting the effect of various dose combinations of oxysterols on M2 cells.

FIG. 5: A) is a bar graph depicting the effect of oxysterols and cytochrome P450 inhibitor SKF525A on M2 cells; B) is a bar graph depicting the effect of oxysterols and cytochrome P450 activator benzylimidazole and inhibitor SKF525A M2 cells.

FIG. 6 is a bar graph depicting the effect of oxysterols on reducing adipogenesis of M2 cells.

FIG. 7: A) are depictions of M2 cell cultures in which adipocytes are visualized by oil Red O stain; B) is a bar graph depicting the number of adipocytes/field in each treatment group; C) is a radiogram of Northern blotting for lipoprotein lipase, adipocyte P2 gene or 18S rRNA in M2 cells exposed to a control or treatment; P) is a bar graph depicting the relative demsometric units of lipoprotein lipase, adipocyte P2 gene mRNA in M2 cells exposed to a control or treatment.

FIG. 8 is a bar graph depicting the effect of synthetic LXR activators on M2 cells.

FIG. 9: A) is a bar graph depicting the effect of COX-1 inhibitor or oxysterol treatment on alkaline phosphatase activity in M2 cells; B) is a bar graph depicting the effect of COX-1 inhibitor or oxysterol treatment on calcium incorporation in M2 cells; C) is a radiogram of Northern blotting for osteoclastin or 18S rRNA in M2 cells exposed to COX-1 inhibitor or oxysterol treatment; D) is a bar graph depicting the relative demsometric units of osteoclastin mRNA in M2 cells exposed to COX-1 inhibitor or oxysterol treatment; E) is a bar graph depicting the effect of PLA₂ inhibitors or oxysterol treatment on alkaline phosphatase activity in M2 cells; and F) is a bar graph depicting the effect of PLA₂ inhibitors or oxysterol treatment on calcium incorporation in M2 cells.

FIG. 10: A) Western blot for pERK or ERK as expressed in M2 cells exposed to control or oxysterol treatment; B) is a bar graph depicting the effect of PD98059 or oxysterol treatment on calcium incorporation in M2 cells; C) is a bar graph depicting the number of adipocytes/field in each treatment group.

FIG. 11 is a table depicting the effect of 22R+20S oxysterol combination on mouse calvaria bone formation.

FIG. 12 are representative sections of calvaria treated with a vehicle (A) or 22R+20S oxysterol (B).

FIG. 13: A) is a bar graph depicting the effect of low dose BMP, oxysterol, or a combination treatment on alkaline phosphatase activity in M2 cells; B) is a bar graph depicting the effect of COX-1 inhibitor or oxysterol treatment on calcium incorporation in M2 cells; C) is a radiogram of Northern blotting for osteoclastin or 18S rRNA in M2 cells exposed to COX-1 inhibitor or oxysterol treatment; D) is a bar graph depicting the relative demsometric units of osteoclastin mRNA in M2 cells exposed to COX-1 inhibitor or oxysterol treatment.

FIG. 14 A) is a bar graph depicting the effect of xanthine/xanthine oxidase, (X; 250 μM/40 mU/ml) inhibition of alkaline phosphatase activity relative to control vehicle (C), and the blockage and reversal by treatment with the oxysterol combination 22S+20S (SS; μM) (*p<0.01 for C vs. X, and for X vs. X+SS at 0.3 and 0.5 μM SS); B) is a Northern blot depicting osteocalcin or 18S rRNA expression after 8 days of treatment with control (Cont.), xanthine/xanthine oxidase or xanthine/xanthine oxidase (XXO) and the oxysterol combination 22S+20S (SS); C) is a bar graph depicting the relative densitometric units of osteocalcin mRNA expression of duplicative samples, such as shown in FIG. 14B).

FIG. 15 A) is a bar graph depicting the effect of minimally oxidized LDL (M; 250 μM/40 mU/ml) inhibition of alkaline phosphatase activity relative to control vehicle (C), and the blockage and reversal by treatment with the oxysterol combination 22S+20S (SS; 2.5, 5, 10 μM) (*p<0.01 for C vs. M, and for M vs. M+SS at all SS concentrations); B) is a Northern blot depicting osteocalcin or 18S rRNA expression after 8 days of treatment with control (Cont.), minimally oxidized LDL (MM) and the oxysterol combination 22S+20S (SS); C) is a bar graph depicting the relative densitometric units of osteocalcin mRNA expression of duplicative samples, such as shown in FIG. 15B).

FIG. 16 is a bar graph depicting the effect of xanthine/xanthine oxidase (XXO; 250 μM/40 mU/ml) or minimally oxidized LDL (MM; 100 μg/ml inhibition of calcium incorporation relative to control vehicle (C), and the blockage and reversal by treatment with the oxysterol combination 22S+20S (SS; 5 μM) (*p<0.01 for C vs. XXO and MM, and for XXO vs. XXO+SS and MM vs. MM+SS).

FIG. 17 A) is a bar graph depicting the effect of 22S+20S (SS; 2.5 μM) protection of the effects of xanthine/xanthine oxidase (XXO; 250 μM/40 mU/ml) or minimally oxidized LDL (MM; 200 μg/ml) inhibition of alkaline phosphatase activity relative to control vehicle (C) or XXO or MM treatment alone; B) is a bar graph depicting the effect of 22S+20S (SS; 2.5 μM) protection of the effects of xanthine/xanthine oxidase (XXO; 250 μM/40 mU/ml) inhibition of calcium incorporation relative to control vehicle (C) or XXO alone; (*p<0.01 for C vs. XXO and MM and for XXO vs. SS/XXO and for MM vs. SS/MM in A, and for C vs. XXO and XXO vs. SS/XXO in B).

FIG. 18 is a bar graph depicting the effect of cyclooxygenase 1 (SC) prevention of 22S+20S (SS; 2.5 μM) protection from the effects of xanthine/xanthine oxidase (X; 250 μM/40 mU/ml) or minimally oxidized LDL (MM; 200 μg/ml) in inhibiting alkaline phosphatase activity relative to control vehicle (C) or SS combination treatments; (*p<0.01 for C vs. MM and X, for MM vs. SS/MM and X vs. SS/X, and for SS/MM vs. SS+SC/MM and SS/X vs. SS+SC/X).

FIG. 19 A) is a bar graph depicting the rescue effect of 22S+20S (SS; 2.5 μM) from the effects of xanthine/xanthine oxidase (XXO; 250 μM/40 mU/ml) or minimally oxidized LDL (MM; 200 μg/ml) inhibition of alkaline phosphatase activity relative to control vehicle (C) or XXO or MM pre-treatment alone; B) is a bar graph depicting the rescue effect of 22S+20S (SS; 2.5 μM) from the effects of xanthine/xanthine oxidase (XXO; 250 μM/40 mU/ml) or minimally oxidized LDL (MM; 200 μg/ml) inhibition of calcium incorporation relative to control vehicle (C) or XXO or MM pre-treatment alone. (*p<0.01 for C vs. XXO and MM, and for XXO vs. XXO/SS and for MM vs. MM/SS in A and B).

FIG. 20 is a radiogram of Northern blotting for osteocalcin mRNA in M2-10B4 cells treated with oxysterols for eight days (5 μM) or control vehicle 1) Control, 2) 4beta-hydroxycholesterol, 3) 24S,25-epoxycholesterol, 4) 7alpha-hydroxycholesterol, and 5) 22S-hydroxycholesterol+20A-hydroxycholesterol.

FIG. 21 A) is a radiogram of a Northern blot for osteocalcin (Osc) and 18S RNA demonstrating the synergistic induction of osteocalcin expression by a combination of oxysterols and BMP7; B) is a radiogram of a Northern blot for osteocalcin (Osc) and 18S RNA demonstrating the synergistic induction of osteocalcin expression by a combination of oxysterols and BMP14.

### DETAILED DESCRIPTION

The present invention is related to agents and methods for inducing osteoblast differentiation, maintaining bone homeostasis, enhancing bone formation and/or enhancing bone repair.

The invention may include the systemic and/or local application of agents for maintaining bone homeostasis, enhancing bone formation and/or enhancing bone repair. Clinical indices of a method or compounds ability to maintain bone homeostasis is evidenced by improvements in bone density at different sites through out the body as assessed by DEXA scanning. Enhanced bone formation in a healing fracture is routinely assessed by regular X-ray of the fracture site at selected time intervals. More advanced techniques for determining the above indices such as quantitative CT scanning may be used.

The invention may include the use of agents which stimulate osteoblastic bone formation. The invention may include the use of agents which influence the differentiation of MSC into osteobalsts.

Agents which may be useful in this invention to affect osteoblastic differentiation include, but are not limited to individual or combinations of oxysterols.

The ability of oxysterols to induce of osteogenic differentiation, mineralization and inhibit adipogenic differentiation may provide a benefit to maintaining bone homeostasis, inducing bone formation or inducing bone repair.

Cholesterol biosynthesis has recently been shown to be involved in marrow stromal cells (MSC) differentiation, as demonstrated by the inhibitory effects of HMG-CoA reductase inhibitors, which could be reversed by mevalonate. Further, oxysterols have been demonstrated to have osteogenic potential as evidenced by their ability to induce osteoblastic differentiation, and additionally mineralization of MSC in vitro. Finally, oxysterols have been demonstrated to have anti-adipogenic effects and inhibit adipocyte differentiation of MSC.

The in vitro models used to show the osteogenic and anti-adipogenic effects of oxysterols are valid and have been used previously in demonstrating similar behaviors of other compounds including bone morphogenetic proteins (BMP). Osteoprogenitor cells including marrow stromal cells (M2 cells), have been shown to act similarly to those present in vivo in animals and humans. These in vitro models have also previously been able to successfully predict the in vivo osteogenic effects of compounds such as BMP and insulin like growth factors (IGF). In addition, the osteogenic effects of the oxysterols in a bone organ culture model using mouse neonatal calvaria have been demonstrated. This organ culture model has also previously been used to successfully predict osteogenic effect of different compounds including BMP in vivo. Therefore, it is anticipated that based on these similar findings, oxysterols will have osteogenic effects in vivo in animals and humans. Demonstration of osteogenic effects of a compound in these in vitro and organ culture models are necessary prior to trials that would demonstrate their effects in vivo in animals and humans.

Oxysterols form a large family of oxygenated derivatives of cholesterol that are present in the circulation and in tissues of humans and animals (Bjorkhem and Diczfalusy 2002. Oxysterols: friends, foes, or just fellow passengers? Arterioscler Thromb Vasc Biol 22:734-742; Edwards and Ericsson 1999. Sterols and isoprenoids: signaling molecules derived from the cholesterol biosynthetic pathway. Annu Rev Biochem 68:157-185; and Schroepfer 2000. Oxysterols: modulators of cholesterol metabolism and other processes. Physiol Rev 80:361-554). They may be formed at least by autooxidation, as a secondary byproduct of lipid peroxidation, or by the action of specific monooxygenases, most of which are members of the cytochrome P450 family of enzymes (Russell 2000. Oxysterol biosynthetic enzymes. Biochim Biophys Acta 1529:126-135.). Oxysterols may also be derived from the diet (Lyons et al. 1999. Rapid hepatic metabolism of 7-ketocholesterol in vivo: implications for dietary oxysterols. J Lipid Res 40:1846-1857). A role for specific oxysterols has been implicated in physiologic and pathologic processes including cellular differentiation, inflammation, apoptosis, steroid production, and atherogenesis (Bjorkhem and Diczfalusy 2002. Oxysterols: friends, foes, or just fellow passengers? Arterioscler Thromb Vasc Biol 22:734-742; Edwards and Ericsson 1999. Sterols and isoprenoids: signaling molecules derived from the cholesterol biosynthetic pathway. Annu Rev Biochem 68:157-185; and Schroepfer 2000. Oxysterols: modulators of cholesterol metabolism and other processes. Physiol Rev 80:361-554). Specific oxysterols, namely a combination of 22R- or 22S- and 20S-hydroxycholesterol, have very potent osteogenic activity (Kha et al. 2004. Oxysterols regulate differentiation of mesenchymal stem cells: pro-bone and anti-fat. J Bone Miner Res 19:830-840). These oxysterol combinations induce the osteoblastic differentiation of a variety of mesenchymal osteoprogenitor cells including the M2 marrow stromal cells, MC3T3-E1 calvarial cells, C3H10T1/2 embryonic fibroblastic cells, and primary mouse bone marrow cells (Kha et al. 2004. Oxysterols regulate differentiation of mesenchymal stem cells: pro-bone and anti-fat. J Bone Miner Res 19:830-840). The osteogenic effects of the oxysterols are believed to be mediated via. COX/PLA2- and MAPK-dependent mechanisms (Kha et al. 2004. Oxysterols regulate differentiation of mesenchymal stem cells: pro-bone and anti-fat. J Bone Miner Res 19:830-840). To the extent oxysterols are also synthetic, they are also of use in this invention.

Agents which may be useful in this invention to effect osteoblastic differentiation include, but are not limited to individual oxysterols such as 22R-, 22S-, 20S, and 25-hydroxycholesterol, pregnanolone, 5-cholesten-3beta, 20alpha-diol 3-acetate (referred to as 20A-hydroxycholesterol), 24-hydroxycholesterol, 24S, 25-epoxycholesterol, 26-hydroxycholesterol, 4 beta-hydroxycholescterol, individually or in combination with each other.

Particular examples of combinations of oxysterols which may be useful in the invention include: 1) 22R- and 20S-hydroxycholesterol, 2) 22S- and 20S-hydroxycholesterol, 3) 22S-hydroxycholesterol+20A-hydroxycholesterol, 4) 22R hydroxycholesterol and 20A-hydroxycholesterol, 5) 22S-hydroxycholesterol and 26-hydroxycholesterol, and 6) 20A-hydroxycholesterol and 20S-hydroxycholesterol.

The invention may further include any portion of the oxysterol molecule which is found to be active in effecting osteoblastic differentiation or bone formation. The invention may further include the activation of a molecule at which the oxysterols are active in affecting osteoblastic differentiation or bone formation. The invention may also include other lipid molecules or analogs designed to mimic the active portions of the above oxysterols, which would act similarly to the parent molecules, via similar mechanisms of action, and similar receptors that would have a positive impact on bone homeostasis.

Mechanism of action. The mechanisms by which oxysterols are physiologically active have been examined, and oxysterols have been shown to be active and effected by a variety of cellular pathways. First, the effects of oxysterols on osteoblastic differentiation have been demonstrated to be potentiated by a cytochrome P450 inhibitor. The effects of oxysterols on osteoblastic differentiation are also mediated by enzymes in the arachidonic acid metabolic pathway, i.e. cyclooxygenase (COX) and phospholipase A2, and ERK. Second, arachidonic acid, released for example from cellular phospholipase activity positively effects the oxysterol effect on osteoblastic differentiation. Third, prostaglandins, including prostaglandin E2 and osteogenic prostanoids, metabolized by the COX enzymes positively effects the oxysterol effect on osteoblastic differentiation. Fourth, extra-cellular signal-regulated kinase (ERK) activity is increased by oxysterols and is correlated with osteoblastic differentiation and mineralization. Therefore, these agents or agents which stimulate the mechanism of oxysterol action may also be useful in this invention.

Further, oxysterols are known to bind to and activate nuclear hormone receptors called liver X receptors (LXR) which then bind to consensus binding sites on the promoters of genes that are regulated by LXR. Additional orphan nuclear hormone receptors may also serve as oxysterol binding sites that could mediate some of the regulatory effects of oxysterols. The invention may include the use of agents which inhibit osteoclastic bone resorption.

The invention includes a medicament for use in the treatment of bone disorders comprising a therapeutically effective dosage of at least one oxysterol selected from the group comprising 20S-hydroxycholesterol, 22S-hydroxycholesterol, 22R-hydroxycholesterol, 25-hydroxycholesterol, pregnanolone, 5-cholesten-3beta, 20alpha-diol 3-acetate (referred to as 20A-hydroxycholesterol), 24-hydroxycholesterol, 24S, 25-epoxycholesterol, 26-hydroxycholesterol, 4beta-hydroxycholesterol, or an active portion of any one of these oxysterols.

Therapeutically effective dose. A therapeutically effective dose of an agent useful in this invention is one which has a positive clinical effect on a patient as measured by the ability of the agent to induce osteoblastic differentiation improve bone homeostasis, bone formation or bone repair, as described above. The therapeutically effective dose of each agent. can be modulated to achieve the desired clinical effect, while minimizing negative side effects. The dosage of the agent may be selected for an individual patient depending upon the route of administration, severity of the disease, age and weight of the patient, other medications the patient is taking and other factors normally considered by an attending physician, when determining an individual regimen and dose level appropriate for a particular patient.

By way of example, the invention may include elevating endogenous, circulating oxysterol levels over the patient's basal level. In normal adult levels are about 10-400 ng/ml depending on age and type of oxysterol, as measured by mass spectrometry. Those skilled in the art of pharmacology would be able to select and monitor the dose to determine if an increase circulating levels over basal levels has occurred.

Dosage Form. The therapeutically effective dose of an agent included in the dosage form may be selected by considering the type of agent selected and the route of administration. The dosage form may include an agent in combination with other inert ingredients, including adjuvants and pharmaceutically acceptable carriers for the facilitation of dosage to the patient, as is known to those skilled in the pharmaceutical arts. In one embodiment, the dosage form may be an oral preparation (ex. liquid, capsule, caplet or the like) which when consumed results in the elevated levels of the agent in the body. The oral preparation may comprise carriers including dilutents, binders, time-release agents, lubricants and disinigrants.

The dosage form may be provided in a topical preparation (ex. lotion, crème, ointment, transdermal patch, or the like) for dermal application. The dosage form may also be provided in preparations for placement at or near the site where osteoblastic differentiation, bone formation or repair is desired, or for subcutaneous (such as in a slow-release capsule), intravenous, intraparitoneal, intramuscular or respiratory application, for example.

Any one or a combination of agents may be included in a dosage form. Alternatively, a combination of agents may be administered to a patient in separate dosage forms. A combination of agents may be administered concurrent in time such that the patient is exposed to at least two agents for treatment, or serially in time such that the patient is exposed to at least two agents for treatment.

Additional Agents. The invention may include treatment with an additional agent which acts independently or synergistically with at least a first agent to maintaining bone homeostasis, enhancing bone formation and/or enhancing bone repair.

Additional agents may be agents which stimulate the mechanistic pathway by which oxysterols enhance osteoblastic differentiation.

BMP has been found to play a role in the differentiation of osteoblasts both in vitro and in vivo. BMP are members of the TGF-beta super family of growth factors and consist of over 10 different proteins. BMP2 and BMP7 have received attention as potential bone anabolic factors. BMP2 is the most potent known inducer of bone formation in vivo, and enhances the differentiation of osteoprogenitor precursor of M2 cells in vitro.

Unexpectedly, oxysterols act in synergy with BMP to induce osteoblastic differentiation and enhance the osteogenic effects of the individual oxysterols (such as 20S-, 22S, 22R-oxysterols) or BMP alone. Further, mineralization has been observed in vitro using combinations of 22R-+20S or 22S-+20S oxysterols and BMP2. Research suggests that although stimulation of MSC by BMP2 can enhance their osteogenic differentiation, the osteogenic effects of the oxysterols do not appear to be a result of the induction of BMP2 expression, as assessed by RT-PCR analysis of BMP2 mRNA in cells treated with a combination of 22R and 20S oxysterols for 4 or 8 days.

Therefore, the invention may include the use of a combination of at least one oxysterol and at least one BMP to induce osteoblastic differentiation, bone homeostasis, formation or repair. This combination of agents to maintain bone homeostasis, enhance bone formation and/or enhance bone repair may be desirable at least in that the dosage of each agent may be reduced as a result of the synergistic effects. In one example, BMP2 may be used for localized use in fracture healing. The dosages used vary. depending on mode of delivery. For example, beads coated with 10-100 micrograms of BMP2 have been used in mouse bone fracture studies. In studies with monkeys, BMP7 has been used in dosages ranging from 500-2000 micrograms. In studies with dogs, BMP2 has been used between 200-2000 micrograms. In studies where BMP2 was delivered in a sponge implanted in the fracture site, the dosage used was 1.5 mg/ml. In a spinal fusion trial where fusion was achieved, a large dose of 10 mg of BMP2 was used. In a human study of tibial non-union fractures in humans, BMP7 was used at several mg dosages.

Additional classes of agents which may be useful in this invention alone or in combination with oxysterols include, but are not limited to cytochrome P450 inhibitors, such as SKF525A. Other classes of agents useful in the invention include phospholipase activators, or arachadonic acid. Other classes of agents useful in the invention include COX enzyme activators, or prostaglandins or osteogenic prostanoids. Other classes of agents useful in the invention include ERK activators.

The invention may include combination treatments with oxysterols and other therapeutics which affect bone formation, repair or homeostasis. For example, oxysterols in combination with bisphosphonates, hormone therapy treatments, such as estrogen receptor modulators, calcitonin, and vitamin D/calcium supplementation PTH (such as Forteo or teriparatide, Eli Lilly), sodium fluoride and growth factors that have a positive effect on bone, such as insulin-like growth factors I and II and transforming growth factor beta. Those skilled in the art would be able to determine the accepted dosages for each of the therapies using standard therapeutic dosage parameters.

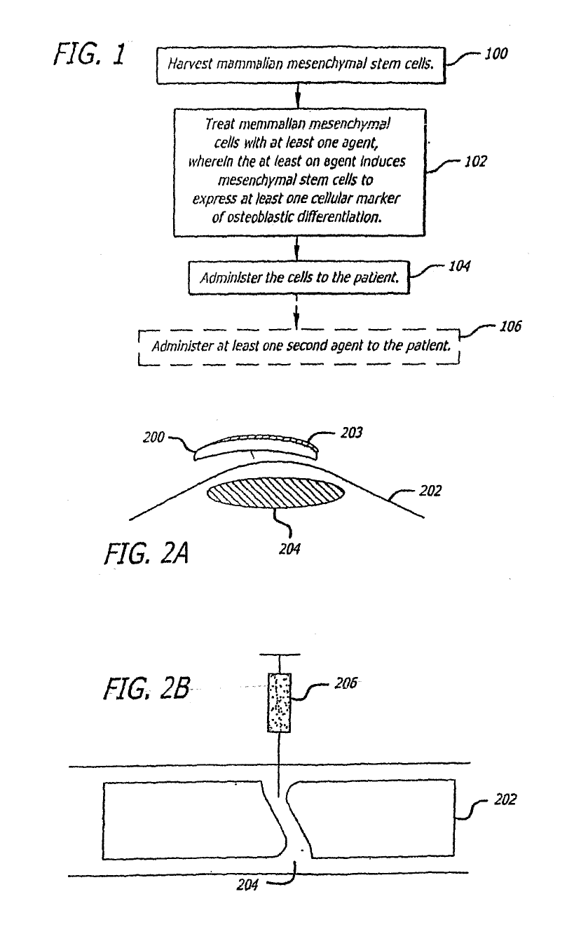

The invention may include a method of systemic delivery or localized treatment with differentiated osteoblastic cells for maintaining bone homeostasis, enhancing bone formation and/or enhancing bone repair. This treatment may be administered alone or in combination with administration of other agent(s) to the patient, as described above. FIG. 1 depicts a flowchart of one method according to this invention. In this embodiment of the method, mammalian mesenchymal stem cells may be harvested, form the patient or a cell donor (100). The cells may then be treated with at least one agent to induce osteoblastic differentiation of the cells (102). The cells may then be re-administered to the patient, either systemically or at a selected site at which bone homeostasis, bone formation or bone repair is desired (104). Additionally, the patient may by treated locally or systemically with. at least one second agent which effects bone homeostasis, bone formation or bone repair (106).

In this aspect of the invention, MSC may be treated with an agent(s) to stimulate osteoblastic differentiation, as measured by any one of the increase in alkaline phosphatase activity, calcium incorporation, mineralization, osteocalcin mRNA expression, Runx2 DNA binding and protein expression, or other indicators of osteoblastic differentiation. In one embodiment of the invention MSC cells are harvested from a patient, treated with at least one oxysterol, and osteoblastic cells are administered to the patient.

The invention may include administering osteoblastically differentiated MSC systemically to the patient.

The invention may include placing osteoblastically differentiated MSC at selected locations in the body of a patient or inducing osteoblastic differentiation with agents including oxysterols after placement. In one embodiment of the invention, cells may be injected at a location at which bone homeostasis, formation and/or repair is desired.

In one application of the invention, the agents and methods may be applied to, but are not limited to the treatment or to slow the progression of bone related disorders, such as osteoporosis.

In applications of the invention, the agents and methods may be applied to, but are not limited to application of cells or agents to a surgical or fracture site, in periodontitis, periodontal regeneration, alveolar ridge augmentation for tooth implant reconstruction, treatment of non-union fractures, sites of knee/hip/joint repair or replacement surgery.

FIG. 2 depicts two embodiments of the present invention. In FIG. 2A, the invention may include implants (200) for use in the human body comprising, a substrate having a surface (201), wherein at least a portion of the surface of the implant includes at least one oxysterol (203) in an amount sufficient to induce osteoblastic differentiation, bone homeostasis, formation or repair in the surrounding tissue, or implant includes mammalian cells capable of osteoblastic differentiation, or osteoblastic mammalian cells, or a combination thereof for inducing bone formation or enhancing bone repair. For example, implants may include, but are not limited to pins, screws, plates or prosthetic joints which may be placed in the proximity of or in contact with a bone (202) that are used to immobilize a fracture, enhance bone formation, or stabilize a prosthetic implant by stimulating formation or repair of a site of bone removal, fracture or other bone injury (204).

As shown in FIG. 2B, the invention may also include the application of at least one agent or differentiated cells (206) in the proximity of or in contact with a bone (202) at a site of bone removal, fracture or other bone injury (204) where bone formation or bone repair is desired.

The invention may include compositions, substrates and methods for the use of a single oxysterol or combination of oxysterols alone to combat oxidative stress. The invention may include the use of a BMP alone or combination with one or more oxysterols alone to combat oxidative stress. More specifically, the oxysterol combination of 22S+20S oxysterols may be used prior to, concurrently with or following oxidative stress caused in part or in whole by agents such as xanthine/xanthine oxidase (XXO) and/minimally oxidized LDL (MM-LDL) (or agents acting by similar molecular mechanisms) to minimize or eliminate the effects of oxidative stress which inhibit osteogenic differentiation, as measured at least by a reduction in alkaline phosphatase activity and/or calcium incorporation by marrow stromal cells. Additionally or alternatively, the rhBMP2 may be used prior to, concurrently with or following oxidative stress caused in part or in whole. by agents such as xanthine/xanthine oxidase (XXO) and/minimally oxidized LDL (MM-LDL) (or agents acting by similar molecular mechanisms) to minimize or eliminate the effects of oxidative stress which inhibit osteogenic differentiation, as measured at least by a reduction in alkaline phosphatase activity and/or calcium incorporation by marrow stromal cells.

### EXAMPLES

Materials: Oxysterols, beta-glycerophosphate (βGP), silver nitrate, oil red O were obtained from Sigma (St. Louis, Mo., U.S.A.), RPMI 1640, alpha modified essential medium (α-MEM), and Dulbecco's modified Eagle's medium (DMEM) from Irvine Scientific (Santa Ana, Calif., U.S.A.), and fetal bovine serum (FBS) from Hyclone (Logan, Utah, U.S.A.). PD98059 was purchased from BIOMOL Research Labs (Plymouth Meeting, Pa., U.S.A.), TO-901317, SC-560, NS-398, Ibuprofen, and Flurbiprofen from Cayman Chemical (Ann Arbor, Mich., U.S.A.), ACA and AACOCF3 from Calbiochem (La Jolla, Calif., U.S.A.), recombinant human BMP2 from R&D Systems (Minneapolis, Minn., U.S.A.). Antibodies to phosphorylated and native ERKs were obtained from New England Biolabs (Beverly, Mass., U.S.A.) and troglitazone from Sankyo (Tokyo, Japan).

Cells: M2-10B4 mouse marrow stromal cell line obtained from American Type Culture Collection (ATCC, Rockville, Md., U.S.A.) was derived from bone marrow stromal cells of a (C57BL/6J x C3H/HeJ) Fl mouse, and support human and murine myelopoiesis in long-term cultures (as per ATCC) and have the ability to differentiate into osteoblastic and adipocytic cells. Unless specified, these cells were cultured in RPMI 1640 containing 10% heat-inactivated FBS, and supplemented with 1 mM sodium pyruvate, 100 U/ml penicillin, and 100 U/ml streptomycin (all from Irvine Scientific).

MC3T3-E1 mouse preosteoblastic cell line was purchased from ATCC and cultured in α-MEM containing 10% heat-inactivated FBS and supplements as described above.

C3H-10T1/2 mouse pluripotent embryonic fibroblast cells were a kindly provided by Dr. Kristina Bostrom (UCLA) and were cultured in DMEM containing 10% heat-inactivated FBS and supplements as described above. Primary mouse marrow stromal cells were isolated from male 4-6 months old C57BL/6J mice, and cultured and propagated as previously reported. Parhami, F. et al., J. Bone Miner. Res. 14, 2067-2078 (1999), herein incorporated by reference in its entirety.

Alkaline phosphatase activity assay: Colorimetric alkaline phosphatase (ALP) activity assay on whole cell extracts was performed as previously described.

Von Kossa and oil red O staining—Matrix mineralization in cell monolayers was detected by silver nitrate staining as previously described. Oil red O staining for detection of adipocytes was performed as previously described.

⁴⁵Ca incorporation assay—Matrix mineralization in cell monolayers was quantified using the ⁴⁵Ca incorporation assay as previously described.

Western blot analysis—After treatments, cells were lysed in lysis buffer, protein concentrations determined using the Bio-Rad protein assay (Hercules, Calif. U.S.A.), and SDS-PAGE performed as previously described. Probing for native and phosphorylated ERKs was performed as previously reported.

RNA isolation and Northern blot analysis—Following treatment of cells under appropriate experimental conditions, total RNA was isolated using the RNA isolation kit from Stratagene (La Jolla, Calif., U.S.A.). Total RNA (10 mg) was run on a 1% agarose/formaldehyde gel and transferred to Duralon-UV membranes (Strategene, CA, U.S.A.) and cross-linked with UV light. The membranes were hybridized overnight at 60 degree C. with ³²P-labeled mouse osteocalcin cDNA probe, mouse lipoprotein lipase (LPL), mouse adipocyte protein 2 (aP²) PCR-generated probes, human 28S or 18S rRNA probes obtained from Geneka Biotechnology (Montreal, Quebec, Canada) and Maxim Biotech (San Francisco, Calif., U.S.A.), respectively. The PCR products were generated using primer sets synthesized by Invitrogen (Carlsbad, Calif., U.S.A.) with the following specifications: mouse aP2 gene (accession no. M13261); sense (75-95) 5′-CCAGGGAGAACCAAAGTTGA-3′, antisense (362-383) 5′-CAGCACTCACCCACTTCTTTC-3′, generating a PCR product of 309 base pairs. Mouse LPL (accession no. XM_{—}134193); sense (1038-1058) 5′-GAATGAAGAAAACCCCAGCA-3′, antisense (1816-1836) 5′-TGGGCCATTAGATTCCTCAC-3′, generating a PCR product of 799 base pairs. The PCR products were gel-purified and sequenced by the UCLA sequencing core, showing the highest similarity to their respective GenBank entries. Following hybridization, the blots were washed twice at room temperature with 2×SSC+0.1% SDS, and then twice at 60 degree C. with 0.5×SSC+0.1% SDS, and exposed to X-ray film. The extent of gene induction was determined by densitometry.

Statistical Analyses—Computer-assisted statistical analyses were performed using the StatView 4.5 program. All p values were calculated using ANOVA and Fisher's projected least significant difference (PLSD) significance test. A value of p<0.05 was considered significant.

### Example A

### Osteogenic Effects of Oxysterols in MSC

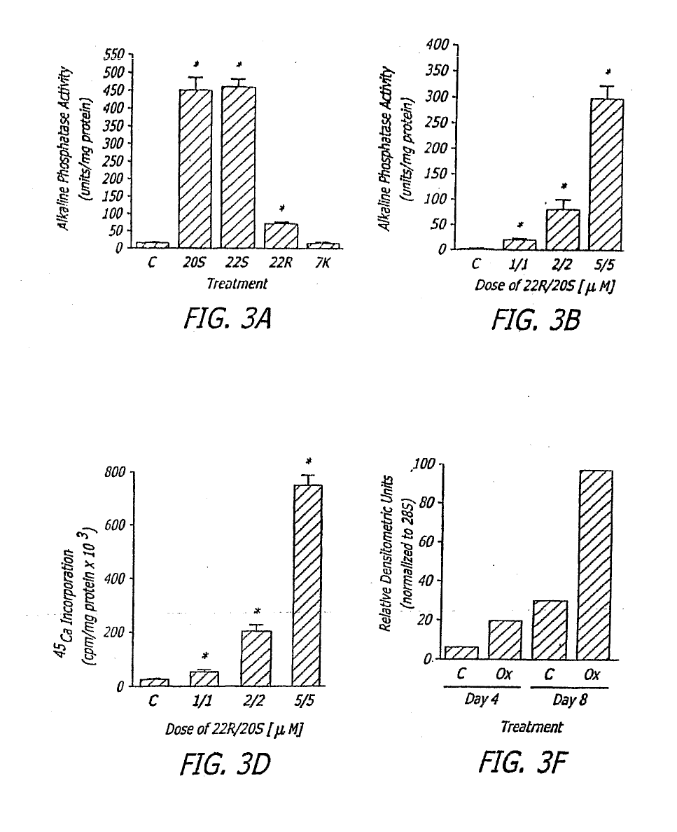

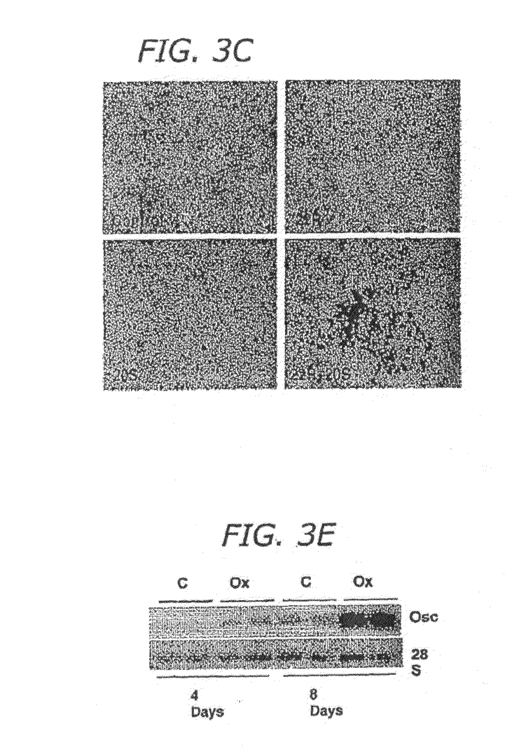

Test 1: M2 cells at confluence were treated with control vehicle (C), or 10 μM oxysterols, in an osteogenic medium consisting of RPMI 1640 to which 10% fetal bovine serum (FBS), 50 μg/ml ascorbate and 3 mM beta-glycerophosphate (μGP) were added. After 3 days of incubation, alkaline phosphatase (ALP) activity was determined in cell homogenates by a colorimetric assay. Results from a representative of five experiments are shown, reported as the mean ±SD of quadruplicate determinations, normalized to protein concentration (* p<0.01 for C vs. oxysterol-treated cells). FIG. 3A is a bar graph depicting the effect of various oxysterols on alkaline phosphatase activity in M2 cells relative to control cells.

M2 cells at confluence were treated in osteogenic medium with control vehicle (C) or a combination of 22R and 20S oxysterols, at the indicated concentrations. ALP activity was measured after 3 days as described above. Results from a representative of four experiments are shown, reported as the mean ±SD of quadruplicate determinations, normalized to protein concentration (* p<0.01 for C vs. oxysterols). FIG. 3B is a bar graph depicting the effect of a combination of oxysterols at various doses on alkaline phosphatase activity in M2 cells.

M2 cells at confluence were treated in osteogenic medium with control vehicle or 5 μM oxysterols, alone or in combination as indicated. After 14 days, mineralization was identified by a von Kossa staining, which appears black. FIG. 3C is a depiction of von Kossa staining of M2 cells exposed to various conditions.

M2 cells were treated with control vehicle (C) or a combination of 22R and 20S oxysterols at increasing concentrations. After 14 days, matrix mineralization in cultures was quantified using a ⁴⁵Ca incorporation assay. Results from a representative of four experiments are shown, reported as the mean ±SD of quadruplicate determinations, normalized to protein concentration (* p<O.01 for C vs. oxysterol-treated cultures). FIG. 3D is a bar graph depicting the effect of-a combination of oxysterols at various doses on calcium incorporation in M2 cells.

M2 cells at confluence were treated with control vehicle (C) or a combination of 22R and 20S oxysterols (5 μLM each) in osteogenic medium. After 4 and 8 days, total RNA from duplicate samples was isolated and analyzed for osteocalcin (Osc) and 28S rRNA expression by Northern blotting as described. FIG. 3E is a radiogram of Northern blotting for osteocalcin mRNA in M2 cells exposed to a control or combination of oxysterols for 4 or 8 days. FIG. 3F is a bar graph depicting the relative demsometric units of osteocalcin mRNA in M2 cells exposed to a control or combination of oxysterols for 4 -or 8 days. Data from densitometric analysis of the Northern blot is shown in (F) as the average of duplicate samples, normalized to 28S rRNA.

Results Test 1: In cultures of MSC, stimulation of alkaline phosphatase activity, osteocalcin gene expression and mineralization of cell colonies are indices of increased differentiation into osteoblast phenotype. Specific oxysterols, namely each of 22R-hydroxycholesterol (22R), 20S-hydroxycholesterol (20S), and 22S-hydroxycholesterol (22S), induced alkaline phosphatase activity, an early marker of osteogenic differentiation, in pluripotent M2-10B4 murine MSC (M2). 7-ketocholesterol (7K) did not induce alkaline phosphatase activity in these cells.

The induction of alkaline phosphatase activity was both dose- and time-dependent at concentrations between 0.5-10 μM, and showed a relative potency of 20S>22S>22R. A 4-hour exposure to these oxysterols followed by replacement with osteogenic medium without oxysterols was sufficient to induce alkaline phosphatase activity in M2 cells, measured after 4 days in culture.

Individual oxysterols (22R, 20S and 22S) at concentrations from 0.5 to less than 10 μM were unable to induce mineralization or osteocalcin gene expression after as many as 14 days of treatment (data not shown). However, alkaline phosphatase activity (FIG. 3B), robust mineralization (FIGS. 3C and D) and osteocalcin gene expression (FIGS. 3E and F) were all induced in M2 cultures by a combination of the 22R+20S or 22S+20S oxysterols, at doses from 1-5 μM for each oxysterol.

Test 2: M2 cells were grown in RPMI medium containing 10% fetal bovine serum (FBS). At confluence, the cells were treated in RPMI containing 5% FBS plus ascorbate at 50 μg/ml and β-glycerophosphate at 3 mM to induce osteoblastic differentiation. Adipogenic differentiation was induced by treating the cells in growth medium plus 10 ˜M troglitazone. A vehicle (C) or oxysterol treatment was applied to cells in a variety of doses (in μM): 20S-hydroxycholesterol, 25-hydroxycholesterol, 22R-hydroxycholesterol; 22S-hydroxycholesterol; 7-ketocholesterol. Cells were always treated at 90% confluence. After 4 days, alkaline phosphatase activity was determined in whole cell lysates and normalized to protein. Alternatively, MSC cultures were prepared and treated with oxysterols as described above. Cells were treated at 90% confluence with the combination of 22R-hydroxycholesterol and 20S-hydroxycholesterol, each at 5 uM, for 4 to 96 hours. The oxysterols where removed and fresh media without oxysterols was added for a total duration of 96 hours. Alkaline phosphatase activity was measured in whole cell extracts and normalized to protein.

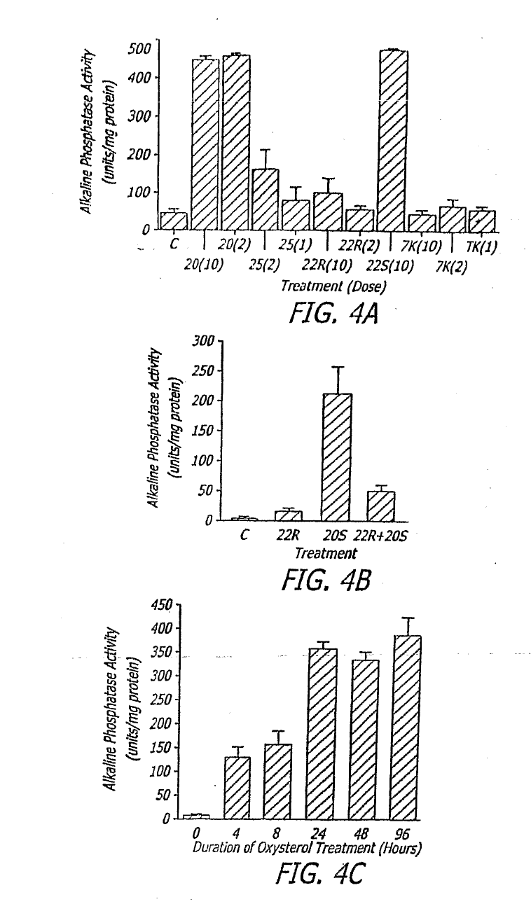

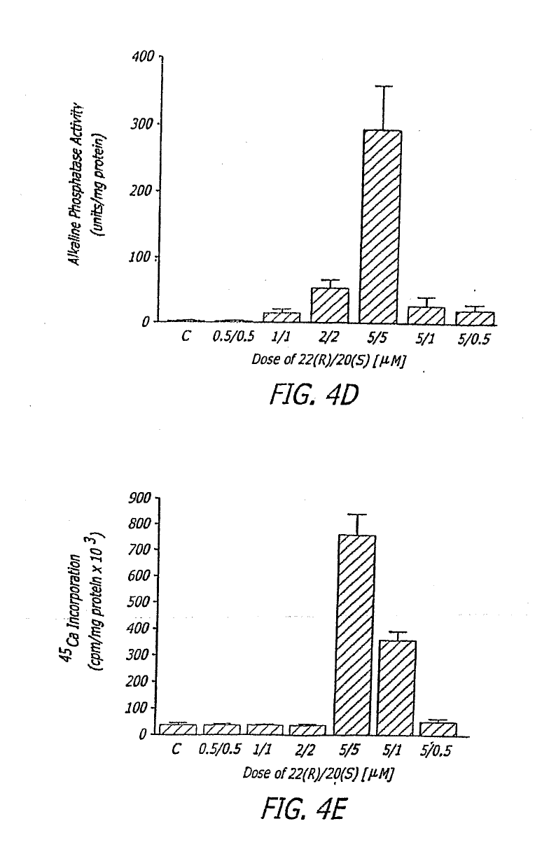

Results Test 2: FIG. 4A is a bar graph depicting the effect of various oxysterols at various doses on M2 cells after 4 days of exposure. Oxysterols induced alkaline phosphatase activity, an early marker of osteoblastic differentiation.

FIG. 4B is a bar graph depicting the effect of various oxysterols at various doses on M2 cells after 24 hours of treatment. Cells were treated at 90% confluence with vehicle (C), or oxysterols 22R-hydroxycholeterol or 20S-hydroxycholesterol, each at 5 μM, alone or in combination. After 24 hours, the cells were rinsed and media replaced with out oxysterols. After 4 days, alkaline phosphatase activity was measured in whole cell extracts and normalized to protein. Alkaline phosphatase activity was induced several fold after only 24 hours of treatment with the oxysterols.

FIG. 4C is a bar graph depicting the effect of duration of treatment with oxysterols on M2 cells. Treatment with a combination oxysterols (22R-hydroxycholesterol and 20S-hydroxycholesterol, each at 5 μM) induced alkaline phosphatase activity after 4-96 hours of treatment as measured 4 days post-treatment.

FIG. 4D is a bar graph depicting the effect of various dose combinations of oxysterols on M2 cells. The effect of the combination oxysterols on M2 cells was dose-dependent for the induction of alkaline phosphatase activity.

FIG. 4E is a bar graph depicting the effect of various dose combinations of oxysterols on M2 cells. Treatment with the combination doses of 22R- and 20S-hydroxycholesterol. After 10 days, ⁴⁵Ca incorporation was measured to assess bone mineral formation, and normalized to protein. The effect of combination oxysterols on M2 cells was dose-dependent for the induction of bone mineral formation as well.

### Example B

### Cytochrome P450 Inhibition of Oxysterol Effects

M2 cells were treated at 90% confluence with vehicle (C), or oxysterols 20S-hydroxycholesterol or 22S-hydroxycholesterol at (0.5 μM) or (1 μM), in the absence or presence of cytochrome P450 inhibitor (SKF525A 10 μM (+)). MSC cultures were also treated at 90% confluence with vehicle (C), or 20S-hydroxycholesterol (2 μM), in the absence or presence of cytochrome P450 activator (benzylimidazole 50 μM) or SKF525A (10 μM). After 4 days, alkaline phosphatase activity was measured in whole cell extracts and normalized to protein.

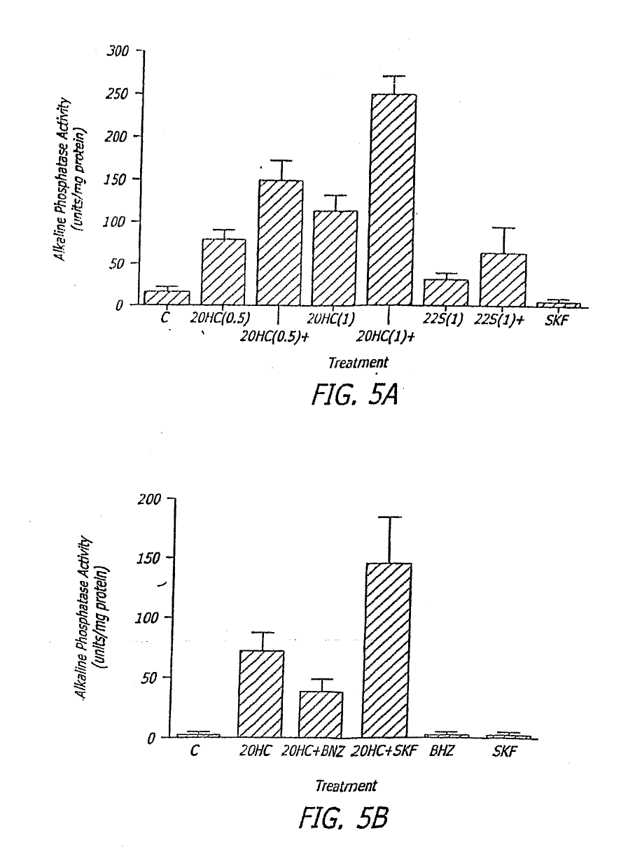

Results Example B: FIG. 5A is a bar graph depicting the effect of oxysterols and cytochrome P450 inhibitor SKF525A on marrow stromal cells. After 4 days, alkaline phosphatase activity was measured in whole cell extracts and normalized to protein. The use of the cytochrome P450 inhibitor potentiated the osteogenic effects of the oxysterols, suggesting that oxysterols are metabolized and inhibited by the cytochrome P450 enzymes.

FIG. 5B is a bar graph depicting the effect of oxysterols and cytochrome P450 activator benzylimidazole and inhibitor SKF525A on M2 cells. Treatment with stimulator of cytochrome P450 enzymes, benzylimidazole, inhibited oxysterol effects, perhaps through enhancing oxysterol degradation.

Example C

### Inhibition of Adipogenesis in MSC by Oxysterols

Adipogenesis of adipocyte progenitors including MSC is regulated by the transcription factor peroxisome proliferator activated receptor γ (PPARγ), that upon activation by ligand-binding, regulates transcription of adipocyte specific genes.

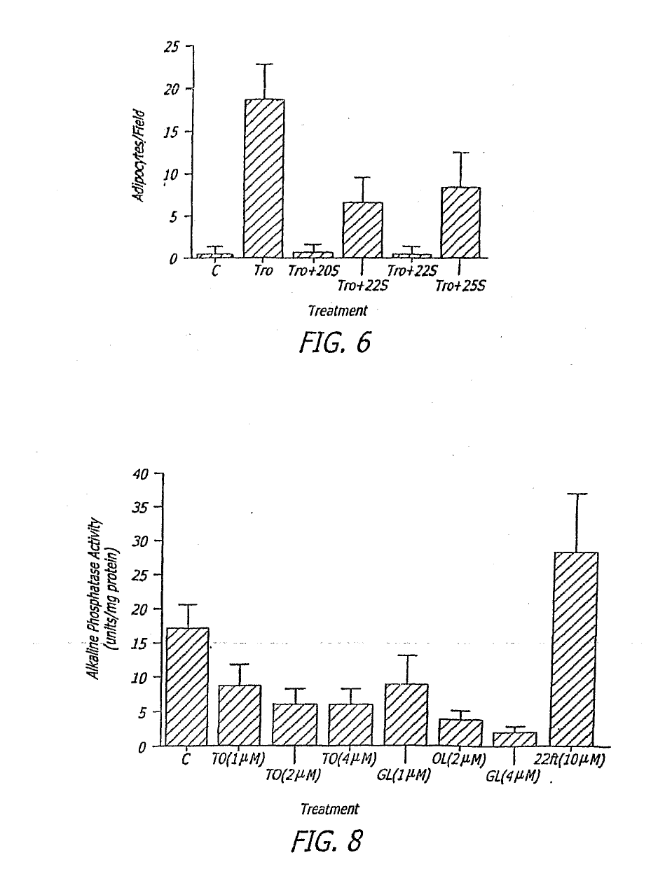

Test 1: M2 cells at 90% confluence were treated with vehicle (C), PPAR-γ activator, troglitazone 10 μM (Tro), alone or in combination with 10 μM oxysterols 20S-, 22R-, or 25S-hydroxycholesterol. After 8 days, adipocytes were identified by oil Red 0 staining and quantified by counting under a phase contrast microscope. FIG. 6A is a bar graph depicting the effect of oxysterols on reducing adipogenesis of MSC. The osteogenic oxysterols inhibited adipogenesis in MSC cultures.

Test 2: (A) M2 cells at confluence were treated in RPMI containing 10% FBS with control vehicle or 10 μM troglitazone (Tro) in the absence or presence of 10 μM 20S or 22S-hydroxycholesterol. After 10 days, adipocytes were visualized by oil Red O staining and quantified by light microscopy, shown in (B). Data from a representative of four experiments are shown, reported as the mean SD of quadruplicate determinations (p<0.001 for Tro vs. Tro+20S and Tro+22S). (C) M2 cells were treated at confluence with 10 μM Tro, alone or in combination with 10 μM 20S oxysterol. After 10 days, total RNA was isolated and analyzed for lipoprotein lipase (LPL), adipocyte P2 gene (aP2) or 18S rRNA expression by Northern blotting as described (Ref). Data from densitometric analysis of the Northern blot is shown in (D) as the average of duplicate samples, normalized to 18S rRNA.

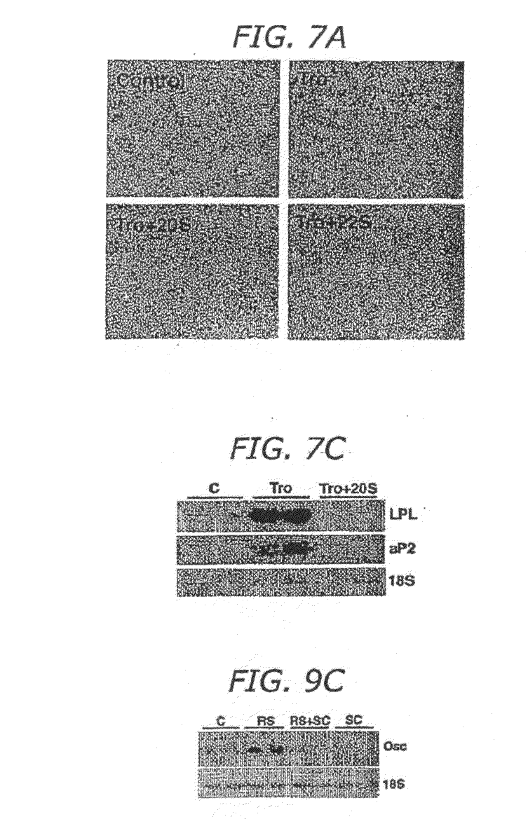

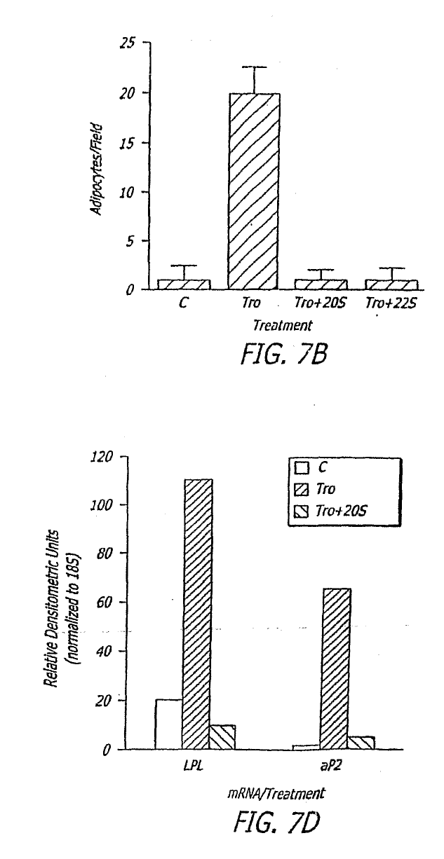

FIG. 7: A) are depictions of M2 cell cultures in which adipocytes are visualized by oil Red O stain; B) is a bar graph depicting the number of adipocytes/field in each treatment group; C) is a radiogram of Northern blotting for lipoprotein lipase, adipocyte P2 gene or 18S rRNA in M2 cells exposed to a control or treatment; D) is a bar graph depicting the relative demsometric units of lipoprotein lipase, adipocyte P2 gene mRNA in M2 cells exposed to a control or treatment.

In M2 cells treated with Tro (PPARγ activator, Troglitazone (Tro)) to induce adipogenesis, 20S, 22S, and 22R-hydroxycholesterol, alone or in combination, inhibited adipogenesis. The relative anti-adipogenic potency of these oxysterols was similar to their relative potency in stimulating alkaline phosphatase activity in M2 cells, with 20S>22S>22R. Similar to its lack of osteogenic effect, 7K was also unable to inhibit adipogenesis in M2 cells (data not shown). Inhibition of adipogenesis was also assessed by an inhibition of the expression of the adipogenic genes lipoprotein lipase (LPL) and adipocyte P2 gene (aP2) by 20S (FIGS. 7C and D). Inhibitory effects of these oxysterols on adipogenesis were also demonstrated using C3H10T1/2 and primary mouse MSC, in which adipogenesis was induced either by Tro or a standard adipogenic cocktail consisting of dexamethasone and isobutylmethylxanthine.

### Example D

### Mechanism of Oxysterol Effects

Liver X receptors (LXR) are nuclear hormone receptors that in part mediate certain cellular responses to oxysterols. LXRα is expressed in a tissue specific manner, whereas LXRβ is ubiquitously expressed. By Northern blot analysis the expression of LXRβ, but not LXRα, in confluent cultures of M2 cells was demonstrated. In order to assess the possible role of LXR in mediating the effects of osteogenic oxysterols, the activation of LXRβ by the pharmacologic. LXR ligand TO-901317 (TO) was examined.

Test 1: TO at 1-10 μM caused a dose-dependent inhibition of alkaline phosphatase activity in M2 cells (C: 18±2; ligands used at 10 μM: 22R=45±5; 20S=140±12; and TO=3±0.5 activity units/mg protein ±SD; p<0.01 for C vs. all ligands). Furthermore, TO treatment did not induce osteocalcin gene expression or mineralization after 10 days. Therefore, the osteogenic effects of the oxysterols on M2 cells thus far appears to be independent of the LXR-beta receptor, as suggested by the potent osteogenic activity of the non-LXR oxysterol ligand 22S and the lack of osteogenic effects in response to the LXR ligand TO.

Test 2: MSC cells at 90% confluence were treated with vehicle (C), or two unrelated LXR ligands (TO and GL at 1-4 μM), or 22R-hydroxycholesterol (10 μM). After 4 days, alkaline phosphatase activity was measured in whole cell lysates and normalized to protein. FIG. 8 is a bar graph depicting the effect of LXR activators on inhibiting osteoblastic differentiation of MSC. LXR-beta is present in MSC, however the osteogenic effects of the oxysterols described above appear not to be through LXR-beta since treatment with specific activators of LXR inhibited osteoblastic differentiation and mineralization of those cells.

### Example E

### Mechanism of Osteogenic Activity of Oxysterols in MSC

Mesenchymal cell differentiation into osteoblasts is regulated by cyclooxygenase (COX) activity. COX-1 and COX-2 are both present in osteoblastic cells, and appear to be primarily involved in bone homeostasis and repair, respectively. Metabolism of arachidonic acid into prostaglandins, including prostaglandin E2 (PGE2), by the COXs mediates the osteogenic effects of these enzymes. COX products and BMP2 have complementary and additive osteogenic effects.

(A) M2 cells at confluence were pretreated for 4 hours with control vehicle (C) or 10 μM COX-1 inhibitor SC-560 (SC) in osteogenic medium as described earlier. Next, a combination of 22R and 20S oxysterols (RS, 2.5 μM each) were added in the presence or absence of SC as indicated. After 3 days, ALP activity was measured as described earlier. Data from a representative of three experiments are shown, reported as the mean ±SD of quadruplicate determinations, normalized to protein concentration (p<0.001 for RS vs. RS+SC). (B) M2 cells were treated as described in (A) and after 10 days matrix mineralization in cultures was quantified by a ⁴⁵Ca incorporation assay as described earlier. Results from a representative of three experiments are shown, reported as the mean ±SD of quadruplicate determinations, normalized to protein concentration. (C) M2 cells were pretreated with 20 μM SC for 4 hours, followed by the addition of RS in the presence or absence of SC as described above. After 8 days, total RNA was isolated and analyzed for osteocalcin (Osc) and 1.8S rRNA expression by Northern blotting as previously described. Data from densitometric analysis of the Northern blot is shown in (D) as the average of duplicate samples, normalized to 18S rRNA. (E) M2 cells at confluence were pretreated for 2 hours with control vehicle (C), or PLA₂ inhibitors ACA (25 μM) and AACOCF3 (AAC, 20 μM), in osteogenic medium. Next, a combination of 22R and 20S oxysterols (RS, 2.5 μM) was added in the presence or absence of the inhibitors as indicated. After 3 days, ALP activity was measured as previously described. Data from a representative of three experiments are shown, reported as the mean ±SD of quadruplicate determinations, normalized to protein concentration (p<0.01 for RS vs. RS+ACA and RSA+AAC). (F) M2 cells were treated as described in (E). After 10 days, matrix mineralization in cultures was quantified using a ⁴⁵Ca incorporation assay as previously described. Results from a representative of three experiments are shown, reported as the mean of quadruplicate determinations ±SD, normalized to protein concentration (p<0.01 for RS vs. RS+ACA and RS+AAC).

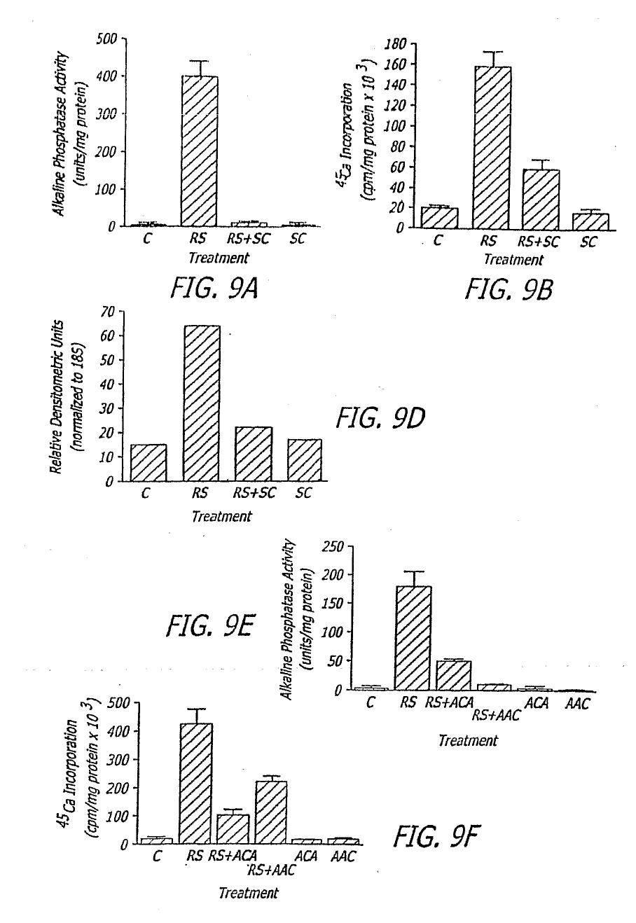

FIG. 9: A) is a bar graph depicting the effect of COX-1 inhibitor or oxysterol treatment on alkaline phosphatase activity in M2 cells; B) is a bar graph depicting the effect of COX-1 inhibitor or oxysterol treatment on calcium incorporation in M2 cells; C) is a radiogram of Northern blotting for osteoclastin or 18S rRNA in M2 cells exposed to COX-1 inhibitor or oxysterol treatment; D) is a bar graph depicting the relative demsometric units of osteoclastin mRNA in M2 cells exposed to COX-1 inhibitor or oxysterol treatment; E) is a bar graph depicting the effect of PLA₂ inhibitors or oxysterol treatment on alkaline phosphatase activity in M2 cells; and F) is a bar graph depicting the effect of PLA₂ inhibitors or oxysterol treatment on calcium incorporation in M2 cells.

In presence of fetal bovine serum, which corresponds to experimental conditions, M2 cells in culture express both COX-1 and COX-2 mRNA at all stages of osteogenic differentiation. Consistent with the role of COX in osteogenesis, the results indicate that the COX-1 selective inhibitor SC-560, at 1-20 μM, significantly inhibited the osteogenic effects of the 22R+20S and 22S+20S oxysterol combinations. SC-560 inhibited oxysterol-induced alkaline phosphatase activity (FIG. 9A), mineralization (FIG. 9B), and osteocalcin gene expression (FIGS. 9C and 9D). Although less effective than SC-560, the non-selective COX inhibitors, ibuprofen and fluriprofin at non-toxic doses of 1-10 μM, also significantly inhibited the osteogenic effects of 22R+20S oxysterol combination by 25-30%. In contrast, the selective COX-2 inhibitor, NS-398, at the highest non-toxic dose of 20 μM had only negligible inhibitory effects.

Furthermore, the osteogenic effects of the oxysterol combination. on alkaline phosphatase activity (FIG. 9E) and mineralization (FIG. 9F) were also inhibited by the general phospholipase A2 (PLA2) inhibitor ACA and by the selective cytosolic PLA2 inhibitor, AACOCF3 (AAC). Activation of PLA2 releases arachidonic acid from cellular phospholipids and makes it available for further metabolism by COX enzymes into prostaglandins. Moreover, rescue experiments showed that the effects of the COX-1 and PLA2 inhibitors on oxysterol-induced alkaline phosphatase activity were reversed by the addition of 1 μM PGE2 and 25 μM arachidonic acid, respectively (data not shown). Consistent with previous reports of oxysterol-stimulated metabolism of arachidonic acid, the present results suggest that the osteogenic activity of the oxysterols in MSC are in part mediated by the activation of PLA2-induced arachidonic acid release, and its metabolism into osteogenic prostanoids by the COX pathway.

### Example F

### Role of ERK in Mediating the Responses of MSC to Oxysterols

The extracellular signal-regulated kinase (ERK) pathway is another major signal transduction pathway previously associated with osteoblastic differentiation of osteoprogenitor-cells. Sustained activation of ERKs mediates the osteogenic differentiation of human MSC52, and activation of ERKs in human osteoblastic cells results in upregulation of expression and DNA binding activity of Cbfa1, the master regulator of osteogenic differentiation. Furthermore, ERK activation appears to be essential for growth, differentiation, and proper functioning of human osteoblastic cells.

(A) M2 cells at confluence were pretreated for four hours with RPMI containing 1% FBS, followed by treatment with control vehicle or 5 μM 20S oxysterol for 1, 4, or 8 hours. Next total cell extracts were prepared and analyzed for levels of native or phosphorylated ERK (pERK) using specific antibodies as previously described. Data from a representative of four experiments are shown, each treatment shown in duplicate samples. (B) M2 cells at confluence were pretreated for 2 hours with control vehicle (C) or 20 μM PD98059 (PD) in osteogenic medium as previously described. Next, a combination of 22R and 20S oxysterols (RS, 5 μM each) were added to appropriate wells as indicated. After 10 days of incubation, matrix mineralization was quantified by the ⁴⁵Ca incorporation assay as previously described. Data from a representative of three experiments are reported as the mean ±SD of quadruplicate determinations, normalized to protein concentration (p<0.01 for RS vs. RS+PD). (C) M2 cells at confluence were pretreated for 2 hours with 20 □M PD98059 (PD) in RPMI containing 5% FBS. Next, the cells were treated with control vehicle (C), 10 μM troglitazone (Tro), or 10 μM of 20S or 22S oxysterols, alone or in combination as indicated. After 10 days, adipocytes were visualized by oil Red O staining and quantified by light microscopy as previously described. Data from a representative of three experiments are reported as the mean ±SD of quadruplicate determinations.

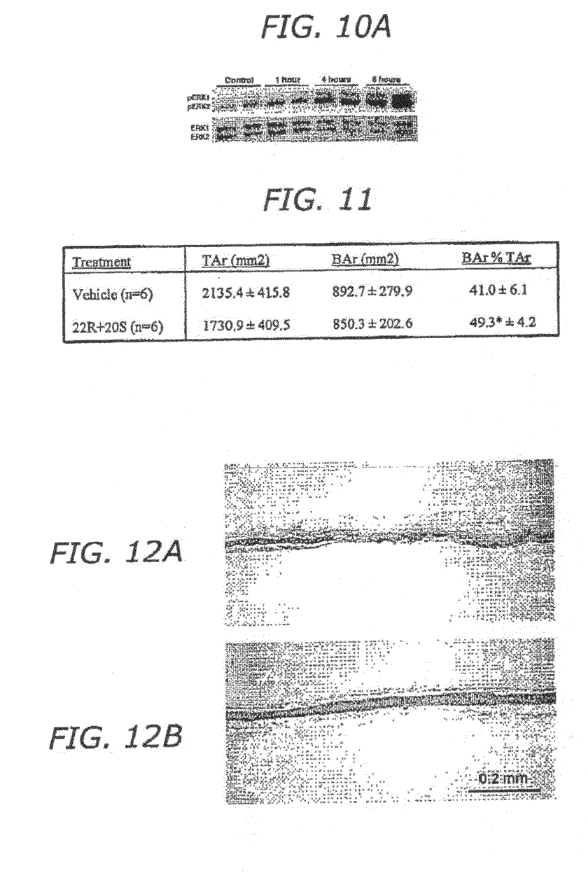

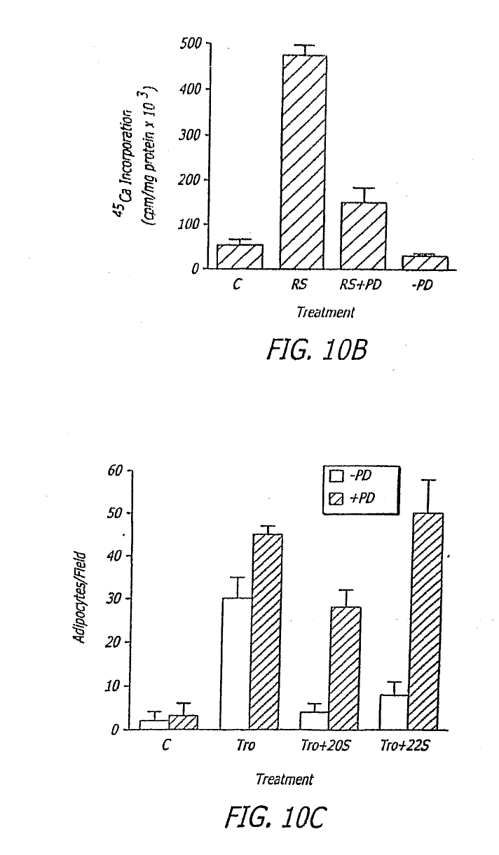

FIG. 10: A) is a Western blot for pERK or ERK as expressed in M2 cells exposed to control or oxysterol treatment; B) is a bar graph depicting the effect of PD98059 or oxysterol treatment on calcium incorporation in M2 cells; C) is a bar graph depicting the number of adipocytes/field in each treatment group

Interestingly, the 20S oxysterol used alone or in combination with 22R oxysterol caused a sustained activation of ERK1 and ERK2 in M2 cells (FIG. 10A). Inhibition of ERK pathway by the inhibitor PD98059,inhibited oxysterol-induced mineralization (FIG. 10B) but not alkaline phosphatase activity or osteocalcin mRNA expression in M2 cell cultures (data not shown). These results suggest that sustained activation of ERK is important in regulating certain specific, but not all, osteogenic effects of oxysterols.

### Example G

The combination of 20S with either 22R or 22S also produced osteogenic effects in the mouse pluripotent embryonic fibroblast C3H10T1/2 cells, in murine calvarial pre-osteoblastic MC3T3-E1 cells, and in primary mouse MSC as assessed by stimulation of alkaline phosphatase activity and mineralization.

Other combinations of oxysterols that had stimulatory effects on osteogenic activity of marrow stromal cells were 22R+pregnanolone, 20S+pregnanolone, both at 5 μM. Pregnanolone is an activator of another nuclear hormone receptor called PXR. However, the most effective combination oxysterols that consistently induced robust osteogenic activity of the marrow stromal cells including both induction of alkaline phosphatase and mineral formation was. 22R- or 22S- in combination with 20S-hydroxycholesterols.

### Example H

Calvaria from 7 days old CD1 pups were surgically extracted (6 per treatment) and cultured for seven days in BGJ medium containing 2% fetal bovine serum in the presence or absence of 22R+20S (5 μM each). Then, the calvaria were prepared and sectioned. Bone area (BAr) and tissue area (TAr) were determined using digital images of H&E stained parietal bones of the calvarial sections. 8-10 images were captured per calvaria, with each image advanced one field of view along the length of the calvaria until the entire section was imaged. The region of analysis extended from the lateral muscle attachments and included the entire calvarial section except for the saggital suture region, which was excluded. The cross sections of the parietal bones were taken approximately equidistant from the coronal and lambdoid sutures and in the same general region for each individual. Sections of this region were analyzed: since they contained little to no suture tissue from the coronal and lambdoid areas. BAr was defined as pink-staining tissue that was not hyper-cellular and displayed a basic lamellar collagen pattern. TAr was defined as the region of tissue between dorsal and ventral layers of lining cells and included BAr as well as undifferentiated cellular tissue and matrix. Separate determinations were made for void area, which was defined as the marrow spaces within the BAr, and was subtracted from BAr measurements prior to calculation of BAr% TAr. To account for differences in TAr between individuals, BAr is reported as a percent of the total TAr measured. Histomorphometric data (continuous variables) were assessed using a one way ANOVA followed by Student's t-test and Dunnett's test vs. control. A p value of 0.05 was used to delineate significant differences between groups. Results are expressed as mean ±SD.

Results. FIG. 11 is a table depicting the effect of 22R +20S oxysterol combination on mouse calvaria bone formation. A 20% increase in bone formation in the calvaria treated with the combination oxysterols was observed compared to those treated with control vehicle, further supporting the osteogenic activity of the combination oxysterols, ex vivo. FIG. 12 are representative sections of calvaria treated with a vehicle (A) or 22R+20S oxysterol

### Example I

### Synergistic Osteogenic Effects of Oxysterols and BMP2 in MSC

(A) M2 cells at confluence were treated with control vehicle (C), 50 ng/ml recombinant human BMP2, or a combination of 22R and 20S oxysterols (RS, 2.5 μM each), alone or in combination in osteogenic medium. ALP activity was measured after 2 days, as described. Results from a representative of four experiments are shown, reported as the mean ±SD of quadruplicate determinations, normalized to protein concentration (p<0.001 for BMP+RS vs. BMP and RS alone). (B) M2 cells were treated as described in (A). After 10 days, matrix mineralization in cultures was quantified using a ⁴⁵Ca incorporation assay as described. Results from a representative of four experiments are shown, reported as the mean ±SD of quadruplicate determinations, normalized to protein concentration (p<0.01 for BMP+RS vs. BMP and RS alone). (C) M2 cells were treated under similar conditions as those described above. After 8 days, total RNA was isolated and analyzed for osteocalcin (Osc) and 18S rRNA expression by Northern blotting as previously described. Data from densitometric analysis of the Northern blot is shown in (D) as the average of duplicate samples, normalized to 18S rRNA.

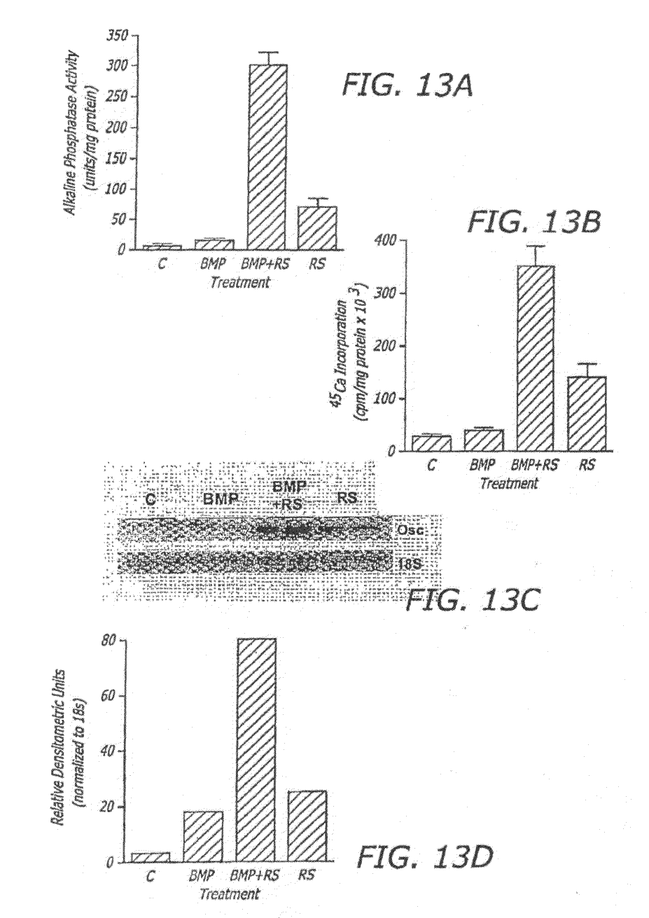

Results. FIG. 13: A) is a bar graph depicting the effect of BMP, oxysterol, or a combination treatment on alkaline phosphatase activity. in M2 cells; B) is a bar graph depicting the effect of COX-1 inhibitor or oxysterol treatment on calcium incorporation in M2 cells; C) is a radiogram of Northern blotting for osteoclastin or 18S rRNA in M2 cells exposed to COX-1 inhibitor or oxysterol treatment; D) is a bar graph depicting the relative demsometric units of osteoclastin mRNA in M2 cells exposed to COX-1 inhibitor or oxysterol treatment. The osteogenic combination of 20S, 22S and 22R oxysterols, as well as the combination of 22R+20S oxysterols acted synergistically with BMP2 in inducing alkaline phosphatase activity (FIG. 13A), the combination of 22R+20S oxysterols acted synergistically with BMP2 induced osteoclastin mRNA expression (FIGS. 13 C & D), and the combination of 22R+20S oxysterols acted synergistically with BMP2 induced mineralization by M2 cells (FIG. 13B).

### Example J

### Inhibition of Osteogenic Differentiation by Oxidative Stress is Blocked and Reversed by Oxysterols

Materials and Methods—Oxysterols, beta-glycerophosphate, ascorbate, xanthine and xanthine oxidase were obtained from Sigma, RPMI 1640 from Irvine Scientific (Santa Ana, Calif. USA), fetal bovine serum (FBS) from Hyclone (Logan, Utah, USA), and SC-560 from Cayman Chemical (Ann Arbor, Mich. USA).

Cell Culture—M2-10B4 mouse marrow stromal cell line (American Type Culture Collection, “ATCC”, Rockville, Md. USA) was derived from bone marrow stromal cells of a (C57BU6J x C3H/HeJ) F1 mouse, and supports human and murine myelopoieses in long-term cultures (as per ATCC). These cells were cultured in RPMI 1640 containing 10% heat-inactivated FBS, and supplemented with 1 mM sodium pyruvate, 100 U/mL penicillin, and 100 U/ml streptomycin (all from Irvine Scientific). The osteogenic medium for these studies consisted of RPMI 1640 with all supplements described above to which 5% FBS, 25 μg/ml ascorbate and 3 mM beta-glycerophosphate were also added.

Lipoprotein preparation and oxidation—Human LDL was isolated by density-gradient centrifugation of serum and stored in phosphate-buffered 0.15 M NaCl containing 0.01% EDTA. Minimally oxidized LDL was prepared by iron oxidation of human LDL, as previously described (Parhami et al. 1999. Atherogenic diet and minimally oxidized low density lipoprotein inhibit osteogenic and promote adipogenic differentiation of marrow stromal cells. J Bone Miner Res 14:2067-2078). The concentrations of lipoproteins used in this study are reported in micrograms of protein. The lipoproteins were tested pre- and post-oxidation for lipopolysaccharide levels and found to have <30 pg of lipopolysaccharide/ml of medium.

Alkaline Phosphatase Activity Assay—Colorimetric alkaline phosphatase activity assay on whole cell extracts was performed as previously described (Kha et al. 2004. Oxysterols regulate differentiation of mesenchymal stem cells: pro-bone and anti-fat. J Bone Miner Res 19:830-840).

⁴⁵Ca Incorporation Assay—Matrix mineralization in cell monolayers was quantified using the ⁴⁵Ca incorporation assay as previously described (Kha et al. 2004. Oxysterols regulate differentiation of mesenchymal stem cells: pro-bone and anti-fat. J Bone Miner Res 19:830-840).

RNA Isolation and Northern Blot Analysis—Total RNA was isolated using the RNA isolation kit from Stratagene (La Jolla, Calif., USA). Northern blotting and analysis of osteocalcin mRNA and 18S rRNA expression was performed as previously described (23).

Statistical Analyses—Computer-assisted statistical analyses were performed using the StatView 4.5 program. All p values were calculated using ANOVA and Fisher's projected least significant difference (PLSD) significance test. A value of p<0.05 was considered significant.

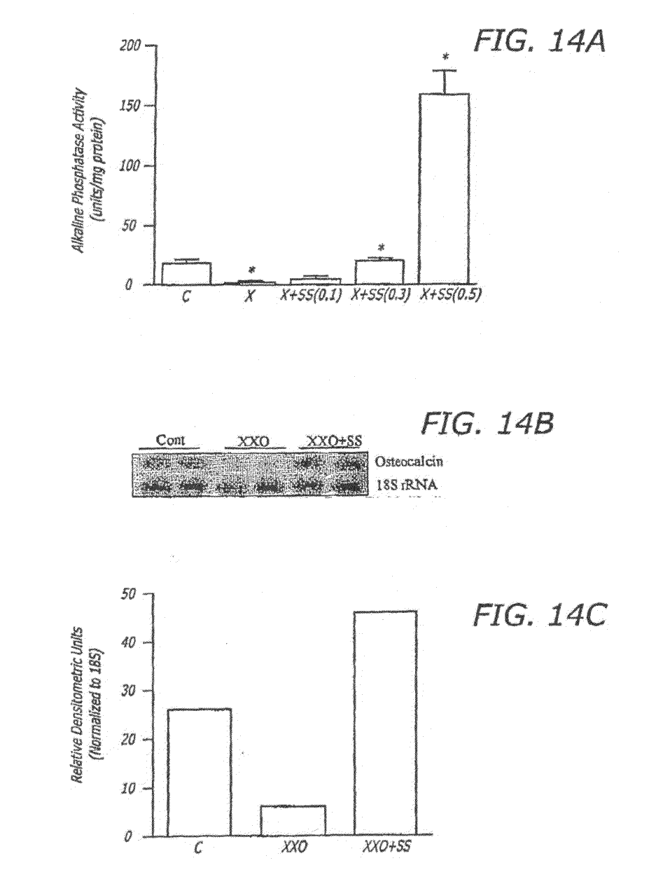

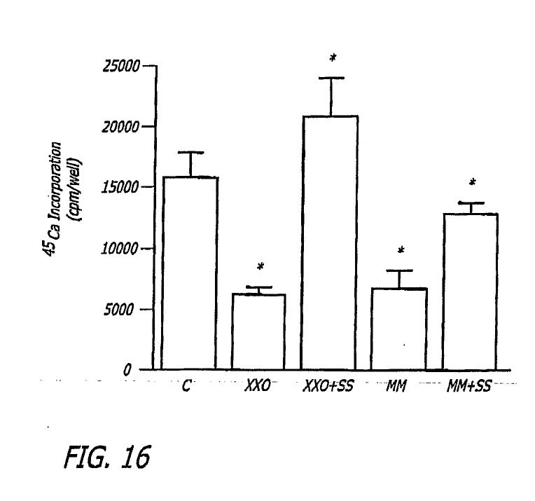

Results—Inhibition of XXO and MM-LDL effects by osteogenic oxysterols. Osteoblastic differentiation of progenitor cells is marked by increased expression of markers including alkaline phosphatase activity, osteocalcin mRNA expression, and mineralization (Rickard et al 1994. Induction of rapid osteoblast differentiation in rat bone marrow stromal cell cultures by dexamethasone and BMP-2. Dev Biol 161:218-228; and Hicok et al. 1998. Development and characterization of conditionally immortalized osteoblast precursor cell lines from human bone marrow stroma. J Bone Miner Res 13:205-217). In order to assess the effect of osteogenic oxysterols on inhibition of osteoblastic differentiation by XXO and MM-LDL, the above differentiation markers were examined in cultures of M2 cells treated with XXO or MM-LDL alone, or in combination with osteogenic oxysterols 22S+20S (SS). Alkaline phosphatase activity was inhibited in M2 cells treated for 6 days with XXO or MM-LDL (FIG. 14A, 15A). Co-treatment with oxysterols (SS) at concentrations of 1.25-5 μM inhibited the effects XXO and MM-LDL in a dose-dependent manner (FIG. 14A, 15A). Inhibition of alkaline phosphatase activity by XXO was blocked by as little as 1.25 μM oxysterols (SS), whereas significant inhibition of MM-LDL effect was achieved with 2.5 μM oxysterols (SS). When M2 cells were cultured in an osteogenic medium, osteocalcin mRNA expression increased with time during osteoblastic differentiation of M2 cells. XXO and MM-LDL inhibited osteocalcin mRNA expression after 8 days, and this inhibition was completely alleviated in the presence of oxysterols (SS) (FIG. 14B, 15B). Furthermore, the inhibitory effect of XXO and MM-LDL on mineralization in cultures of M2 cells was also alleviated in the presence of oxysterols (SS) (FIG. 16). Altogether, these results demonstrate that osteogenic oxysterols inhibit the adverse effects of at least two factors, XXO and MM-LDL, which cause oxidative stress in M2 cells and inhibit their osteogenic differentiation.

Finally, the correlation between protective capacity against oxidative stress and induction of osteogenic differentiation was also demonstrated in the case of rhBMP2. Pretreatment of M2 cells for 48 hours with rhBMP2 (250 ng/ml) rendered these cells protected from the adverse effects of oxidative stress on their osteogenic differentiation (data not shown).

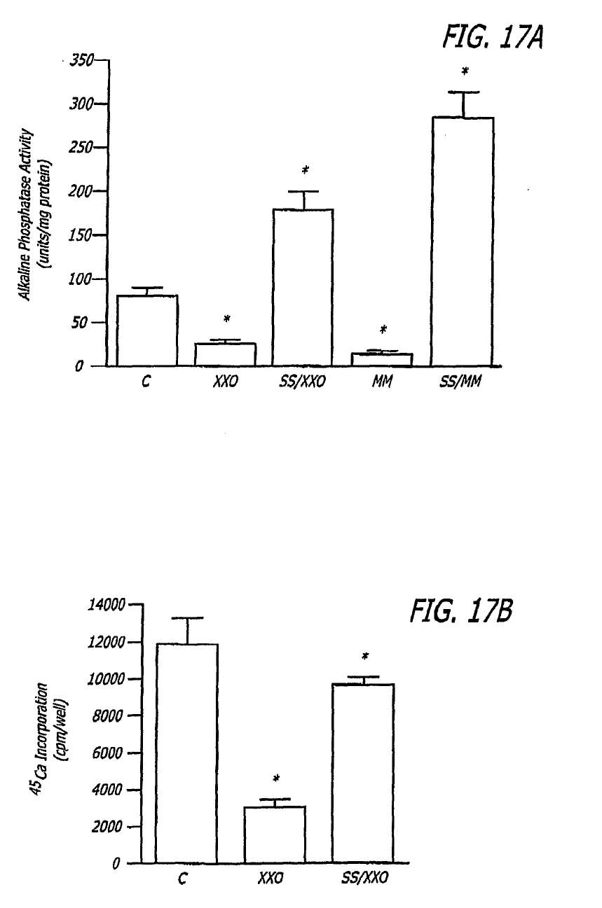

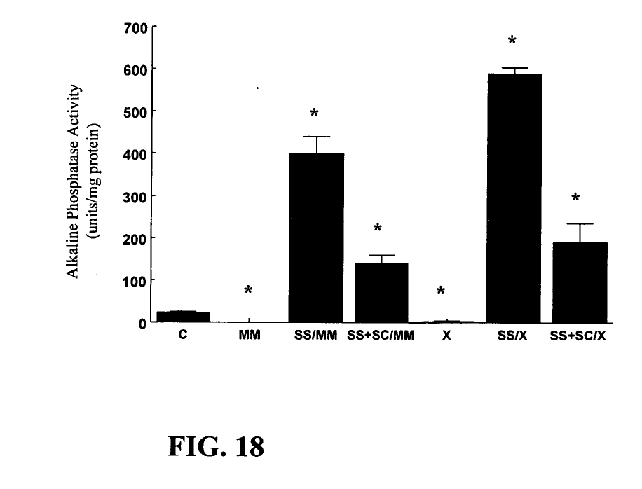

Osteogenic oxysterols protect against the effects of XXO and MM-LDL. In order to examine whether in addition to blocking the inhibitory effects of XXO and MM-LDL on the expression of osteogenic differentiation markers in M2 cells, pretreatment of M2 cells with osteogenic oxysterols can protect these cells from oxidative stress, M2 cells were pretreated for 48 hours with 2.5 μM oxysterols (SS). After 48 hours, oxysterols (SS) was removed and XXO or MM-LDL was added to cells that were pretreated with oxysterols (SS) or control vehicle. Alkaline phosphatase activity was measured after 6 days. In contrast to cells pretreated with control vehicle, in which alkaline phosphatase activity was inhibited by oxidative stresses, cells pretreated with the oxysterols were completely protected from the inhibitory effects of both XXO and MM-LDL (FIG. 17A). Similarly, protective effects of oxysterols (SS) were found on mineralization (FIG. 17B). The protective effects of the osteogenic oxysterols appear to dependent on COX-1, since cells pretreated with SS and COX-1 inhibitor SC-560 were no longer protected against the adverse effects of XXO and MM-LDL (FIG. 18).

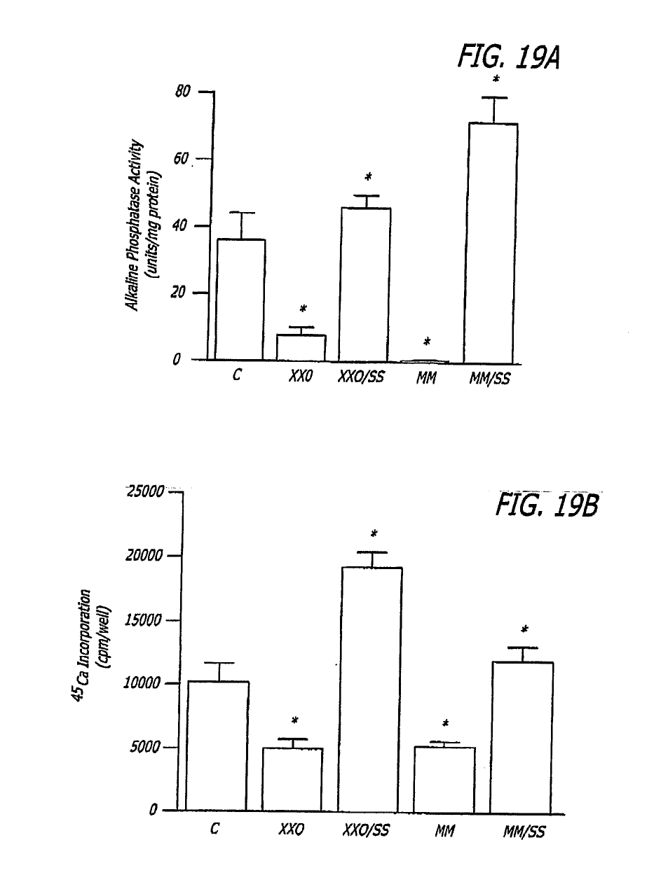

Osteogenic oxysterols rescue cells from the effects of XXO and MM-LDL. Finally, the ability of osteogenic oxysterols to rescue the cells from the inhibitory effects of oxidative stress was examined. M2 cells were pretreated with MM-LDL or XXO for 2 days, followed by their removal and addition of oxysterols (SS) or control vehicle for an additional 4 or 12 days, after which alkaline phosphatase activity and mineralization, respectively, were measured. Results showed that alkaline phosphatase activity (FIG. 19A) and mineralization (FIG. 19B) were inhibited in cells treated for 2 days with MM-LDL or XXO, and that the addition of oxysterols (SS) rescued the cells from the adverse effects of MM-LDL and XXO.

### Example K

The effect of oxysterols on xanthine/xanthine oxidase inhibition of osteoblast marker expression in marrow stromal cells was tested. (A) M2 cells grown to confluence were treated in osteogenic medium with control vehicle (C), xanthine/xanthine oxidase (X; 250 μM/40 mU/ml) or the oxysterol combination 22S+20S (SS; 0.1, 0.3 or 0.5 μM), alone or in combination. After 6 days, alkaline phosphatase activity in whole cell extracts was measured for each treatment group. Results from a representative of 3 separate experiments are reported as the mean ±SD of quadruplicate determinations, normalized to protein concentrations. Further, M2 cells at confluence were treated in osteogenic medium with control vehicle (Cont), xanthine/xanthine oxidase (XXO; 250 μM/40 mU/ml, or oxysterols (SS) (5 μM), alone or in combination. After 8 days, total RNA from duplicate samples was isolated and analyzed for osteocalcin or 18S rRNA expression by Northern blotting. Data from densitometric analysis of the Northern blot are shown as the average of duplicate samples, normalized to 18S rRNA.

Results. FIG. 14 A) is a bar graph depicting the effect of xanthine/xanthine oxidase (X; 250 μM/40 mU/ml) inhibition of alkaline phosphatase activity relative to control vehicle (C), and the blockage and reversal by treatment with the oxysterol combination 22S+20S (SS; μM) (*p<0.01 for C vs. X, and for X vs. X+SS at 0.3 and 0.5 μM SS); B) is a Northern blot depicting osteocalcin or 18S rRNA expression after 8 days of treatment with control (Cont.), xanthine/xanthine oxidase or xanthine/xanthine oxidase (XXO) and the oxysterol combination 22S+20S (SS); C) is a bar graph depicting the relative densitometric units of osteocalcin mRNA expression of duplicative samples, such as shown in FIG. 14B).

### Example L

The effect of oxysterols on minimally oxidized LDL inhibition of osteoblast marker expression in marrow stromal cells was tested. M2 cells at confluence were treated in osteogenic medium with control vehicle (C), minimally oxidized LDL (M; 200 μg/ml) or the oxysterol combination 22S+20S (SS; μM), alone or in combination. After 6 days, alkaline phosphatase activity in whole cell extracts was measured. Results from a representative of 3 separate experiments are reported as the mean ±SD of quadruplicate determinations, normalized to protein concentrations. Further, M2 cells at confluence were treated in osteogenic medium with control vehicle (Cont), minimally oxidized LDL (MM; 200 μg/ml), or oxysterols (SS) (5 μM), alone or in combination. After 8 days, total RNA from duplicate samples was isolated and analyzed for osteocalcin or 18S rRNA expression by Northern blotting. Data from densitometric analysis of the Northern blot are shown as the average of duplicate samples, normalized to 18S rRNA.

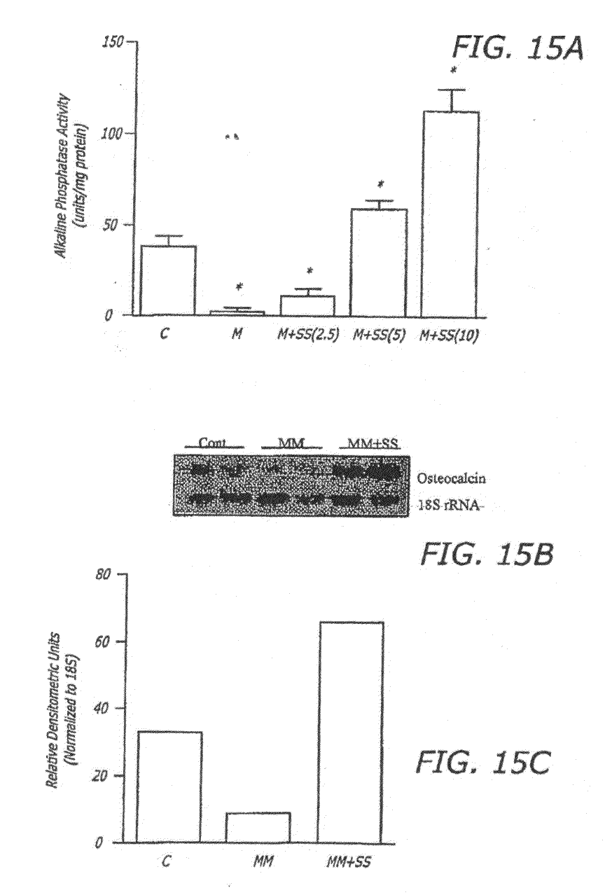

Results. FIG. 15 A) is a bar graph depicting the effect of minimally oxidized LDL (M; 250 μM/40 mU/ml) inhibition of alkaline phosphatase activity relative to control vehicle (C), and the blockage and reversal by treatment with the oxysterol combination 22S+20S (SS; 2.5, 5, 10 μM) (*p<0.01 for C vs. M, and for M vs. M+SS at all SS concentrations); B) is a Northern blot depicting osteocalcin or 18S rRNA expression after 8 days of treatment with control (Cont.), minimally oxidized LDL (MM) and the oxysterol combination 22S+20S (SS); C) is a bar graph depicting the relative densitometric units of osteocalcin mRNA expression of duplicative samples, such as shown in FIG. 15B).

### Example M

The effect of oxysterols on inhibition of mineralization in marrow stromal cells was tested. M2 cells were plated at 20,000 cells per cm², 4 wells per condition, and treated at confluence in osteogenic medium with control vehicle (C), xanthine/xanthine oxidase (XXO; 250 μM/40 mU/ml), minimally oxidized LDL (MM; 100 μg/ml), or SS (5 μM), alone or in combination. After 14 days, matrix mineralization in cultures was quantified using a ⁴⁵Ca incorporation assay. Results from a representative of 3 separate experiments are shown, reported as the mean ±SD of quadruplicate determinations.

Results. FIG. 16 is a bar graph depicting the effect of xanthine/xanthine oxidase (XXO; 250 μM/40 mU/ml) or minimally oxidized LDL (MM; 100 μg/ml inhibition of calcium incorporation relative to control vehicle (C), and the blockage and reversal by treatment with the oxysterol combination 22S+20S (SS; 5 μM) (*p<0.01 for C vs. XXO and MM, and for XXO vs. XXO+SS and MM vs. MM+SS).

### Example N

The protection of marrow stromal cells by oxysterols against the inhibitory effects of xanthine/xanthine oxidase and minimally oxidized LDL on osteoblast marker expression was tested. M2 cells at confluence were pretreated with control vehicle (C) or the oxysterol combination 22S+20S (SS; 2.5 μM) for 48 hours. Next, oxysterols (SS) were removed, cells rinsed, and xanthine/xanthine oxidase (XXO; 250 μM/40 mU/ml) or minimally oxidized LDL (MM; 200 μg/ml) was added in osteogenic medium. After 5 or 14 days of treatment with XXO or MM, (A) alkaline phosphatase activity and (B) mineralization were measured after 5 and 14 days, respectively, as previously described. Results from a representative of three separate experiments are reported as the mean ±SD of quadruplicate determinations.

Results. FIG. 17 A) is a bar graph depicting the effect of 22S+20S (SS; 2.5 μM) protection of the effects of xanthine/xanthine oxidase (XXO; 250 μM/40 mU/ml) or minimally oxidized LDL (MM; 200 μg/ml) inhibition of alkaline phosphatase activity relative to control vehicle (C) or XXO or MM treatment alone; B) is a bar graph depicting the effect of 22S+20S (SS; 2.5 μM) protection of the effects of xanthine/xanthine oxidase (XXO; 250 μM/40 mU/ml) inhibition of calcium incorporation relative to control vehicle (C) or XXO alone; (*p<0.01 for C vs. XXO and MM and for XXO vs. SS/XXO and for MM vs. SS/MM in A, and for C vs. XXO and XXO vs. SS/XXO in B).

### Example O

The effect of cyclooxygenase 1 inhibitor on protection of marrow stromal cells by oxysterols was tested. M2 cells at confluence were pretreated with control vehicle (C) or cyclooxygenase 1 inhibitor, SC-560 (SC; 20 μM) for 2 hours. Next, the oxysterol combination 22S+20S (SS; 2.5 μM) was added. After 48 hours of treatment, SS and SC were removed, the cells rinsed and minimally oxidized LDL (MM; 200 μg/ml) or xanthine/xanthine oxidase (X; 250 μM/40 mU/ml) was added. After 5 days of treatment with MM or X, alkaline phosphatase activity in cell extracts was measured. Results from a representative of 3 separate experiments are reported as the mean ±SD of quadruplicate determinations.

Results. FIG. 18 is a bar graph depicting the effect of cyclooxygenase 1 (SC) prevention of 22S+20S (SS; 2.5 μM) protection from the effects of xanthine/xanthine oxidase (X; 250 μM/40 mU/ml) or minimally oxidized LDL (MM; 200 μg/ml) in inhibiting alkaline phosphatase activity relative to control vehicle (C) or SS combination treatments; (*p<0.01 for C vs. MM and X, for MM vs. SS/MM and X vs. SS/X, and for SS/MM vs. SS+SC/MM and SS/X vs. SS+SC/X).

### Example P

The oxysterol rescue of marrow cells from the inhibitory effects of xanthine/xanthine oxidase and minimally oxidized LDL on osteoblast marker expression was tested. M2 cells at confluence were pretreated for 2 days with control vehicle (C), xanthine/xanthine oxidase (XXO; 250 μM/40 mU/ml) or minimally oxidized LDL (MM; 200 μg/ml) in osteogenic medium. Next, XXO and MM were removed and vehicle or the combination of 22S+20S oxysterols (SS; 2.5 μM) was added. Alkaline phosphatase activity (A) and mineralization (B) were measured after 4 and 12 days of treatment with oxysterols (SS), respectively. Results from a representative of three separate experiments are reported as the mean ±SD of quadruplicate determinations.

Results. FIG. 19 A) is a bar graph depicting the rescue effect of 22S+20S (SS; 2.5 μM) from the effects of xanthine/xanthine oxidase (XXO; 250 μM/40 mU/ml) or minimally oxidized LDL (MM; 200 μg/ml) inhibition of alkaline phosphatase activity relative to control vehicle (C) or XXO or MM pre-treatment alone; B) is a bar graph depicting the rescue effect of 22S+20S (SS; 2.5 μM) from the effects of xanthine/xanthine oxidase (XXO; 250 μM/40 mU/ml) or minimally oxidized LDL (MM; 200 μg/ml) inhibition of calcium incorporation relative to control vehicle (C) or XXO or MM pre-treatment alone. (*p<0.01 for C vs. XXO and MM, and for XXO vs. XXO/SS and for MM vs. MM/SS in A and B).

### Example Q

The purpose of the study was to identify other osteogenic and anti-adipogenic oxysterols based on the chemical structure of previously identified oxysterols. The ability of such candidate oxysterol molecules to induce the formation of osteoblastic cells in cultures of marrow stromal cells (which are progenitors of osteoblastic cells that make bone) were tested. In order to assess osteogenic differentiation of cells, one or more markers of osteogenic differentiation were measured in untreated cells and cells treated with the test oxysterols. These markers included: alkaline phosphatase activity, osteocalcin mRNA expression and mineral formation in cultures of marrow stromal cells. Activation of either one or more than one marker by a single or combination oxysterols is indicative of their osteogenic property.

Method. Cells were treated with the oxysterols(5 μM) for 4 days after which they were collected and analyzed by calorimetric assay for alkaline phosphatase activity. Results from a representative experiment are shown as the average of quadruplicate determination ±SD. Also, cells were treated with the oxysterols for 14 days after which the amount of mineral formed in the cultures was quantified using a radioactive ⁴⁵Ca incorporation assay. Results from a representative experiment are shown as the average of quadruplicate determination ±SD.

Results. By the above methodology, the following new oxysterols were identified as osteoinductive when used either alone or in combination with any of the originally described oxysterols. These asteogenic oxysterols also have anti-adipogenic properties as shown above. The newly identified oxysterols are: 5-cholesten-3beta, 20alpha-diol 3-acetate (referred to as 20A-hydroxycholesterol), 24(S)-hydroxycholesterol, 24S, 25-epoxycholesterol, 26-hydroxycholesterol and 4 beta-hydroxycholestreol.

TABLE 1
Effect of oxysterols on alkaline phosphatase
activity in M2-10B4 marrow stromal cells.

| | Enzyme Activity (units/mg |
|---|---|
| Oxysterol | total cellular protein) ± SD |
| Untreated | 6 ± 3 |
| 20A-hydroxycholesterol | 813 ± 15 |
| 24-hydroxycholesterol | 250 ± 20 |
| 26-hydroxycholesterol | 655 ± 93 |
| 24S,25-epoxycholesterol | 1,588 ± 19 |

TABLE 2
Effect of oxysterols on mineralization
in M2-10B4 marrow stromal cells.

| | ⁴⁵Ca Incorporation (cpm/mg |
|---|---|
| Oxysterol | protein × 10³) ± SD |
| Untreated | 59 ± 25 |
| 22S + 20A (5 μM) | 558 ± 40 |
| 22R + 20A (5 μM) | 2,545 ± 174 |
| 22S + 26-OH (5 μM) | 1,128 ± 129 |
| 20A + 20S (5 μM) | 1,574 ± 913 |

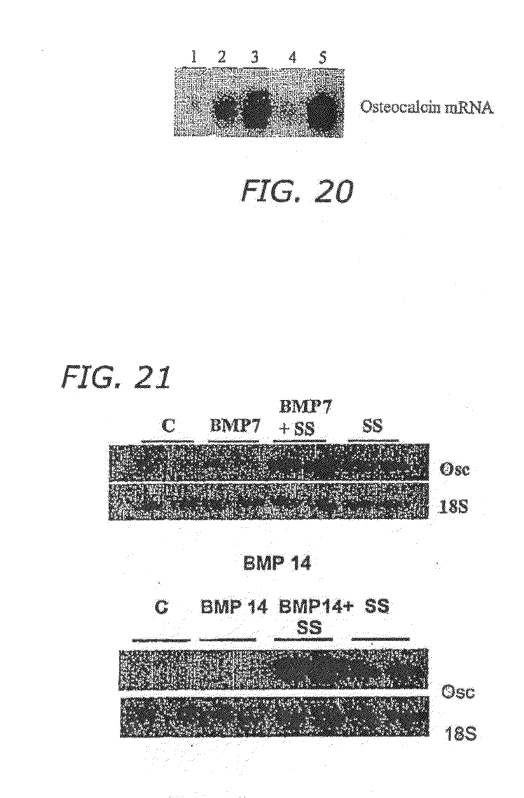

Method: M2-10B4 cells were treated with oxysterols for eight days with oxtsterol (5 μM) or control vehicle. Cells Were harvested, and mRNA extracted. FIG. 20 is a radiogram of Northern blotting for osteocalcin mRNA in M2-10B4cells treated with oxysterols for eight days (5 μM) or control vehicle 1) Control, 2) 4beta-hydroxycholesterol, 3) 24S,25-epoxycholesterol, 4) 7alpha-hydroxycholesterol, and 5) 22S-hydroxycholesterol+20A-hydroxycholesterol.

Results. As shown in FIG. 20, relative to the control, 24S,25-epoxycholesterol, and 22S-hydroxycholesterol+20A-hydroxycholesterol increased the levels of osteocalcin mRNA. 4beta-hydroxycholesterol showed a moderate increase in osteocalcin mRNA over the control group, While 7alpha-hydroxycholesterol did not appear to significantly affect levels of osteocalcin mRNA. This suggests that the carbon side chain of the sterols and the position of the hydroxyl groups may by important characteristics of the osteogenic sterols.

Other cholesterol derivatives, which share some structural similarity to oxysterols (estren, estrone and beta-estradiol) but do not have the carbon side chain and have differences in the position and number of their double bonds, did not have any osteogenic properties.

### Example R

Synergistic osteogenic effects of oxysterols and BMP7 or BMP14 in MSC. A) Marrow stromal cells were treated with control vehicle (C), BMP7 (50 ng/ml), or 22S+20S oxysterol combination (SS, 2.5 μM), alone or in combination. After eight days, RNA was extracted and analyzed for osteocalcin (Osc) or 18S rRNA expression by Northern blotting; B) Marrow stromal cells were treated with control vehicle (C), BMP14 (50 ng/ml), or 22S+20S oxysterol combination (SS, 2.5 μM), alone or in combination. After eight days, RNA was extracted and analyzed for osteocalcin (Osc) or 18S rRNA expression by Northern blotting.

Results. FIG. 21: A) is a radiogram of a Northern blot for osteocalcin (Osc) and 18S RNA demonstrating the synergistic induction of osteocalcin expression by a combination of oxysterols and BMP7; B) is a radiogram of a Northern blot for osteocalcin (Osc) and 18S RNA demonstrating the synergistic induction of osteocalcin expression by a combination of oxysterols and BMP14.

Osteogenic oxysterols synergistically act with BMP7 and BMP14 to induce osteogenic differentiation as evidenced by the synergistic induction of osteogenic differentiation marker osteocalcin shown. Other markers of osteogenic differentiation, alkaline phosphatase activity and mineralization, were also synergistically induced by oxysterols and BMP7 and BMP14.

### Example S

Method: Marrow stromal cells (M2-10B4 (M2)) were treated with doses of 5-15 μM 20S-hydroxycholestrol.

Result. Treatment induced the formation of matrure osteoblastic cells as demonstrated by the induction of alkaline phosphatase (ALP) activity, Runx2 DNA binding and protein expression, osteocalcin mRNA expression and mineralization.

Method: Marrow stromal cells (M2-10B4 (M2)) were treated with doses of 5-15 μM20S-hydroxycholestrol. Groups of cells were pre-treated with 22S or 22R.

Result. Pretreatment and co-treatment of cells with 22S or 22R oxysterols greatly enhanced the osteogenic effects of 20S-hydroxycholesterol's induction of alkaline phosphatase (ALP) activity, Runx2 DNA binding and protein expression, osteocalcin mRNA expression and mineralization. This result suggests that the 22S and 22R prime the osteoprogentitor cells for better responsiveness to the 20S oxysterol.

### Example T

Method: Marrow stromal cells (M2-10B4 (M2)) were pretreated with hedgehog signaling inhibitor, cyclopamine (1-10 μM), then treated with 20S-hydroxycholestrol and 22(S)-hydroxycholesterol.

Result. Pretreatment of the M2 cells with hedgehog signaling inhibitor, cyclopamine inhibited the SS-induced alkaline phosphatase (ALP) activity, Runx2 DNA binding and protein expression, osteocalcin mRNA expression and mineralization. This suggests that the oxysterol induced osteogenic differentiation is mediated through a hedgehog dependant mechanism.

### Example U

Method: Marrow stromal cells (M2-10B4 (M2)) were pretreated with Wnt signaling inhibitor, DKK-1 (1 μg/ml), then treated with 20S-hydroxycholestrol and 22(8)-hydroxycholesterol.

Result. Pretreatment of the M2 cells with Wnt signaling inhibitor inhibited the SS-induced alkaline phosphatase (ALP) activity, osteocalcin mRNA expression and mineralization, but not Runx2 protein expression. This suggests that the oxysterol induced osteogenic differentiation is mediated through a Wnt-dependant mechanism.

1. A method of inducing osteoblastic differentiation and/or inhibiting adipocyte differentiation of mammalian mesenchymal stem cells comprising treating mammalian mesenchymal cells with at least one agent, wherein the agent is selected from the group comprising 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol or a portion of any one of 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol active in inducing osteoblastic differentiation.

2. The method of claim 1, wherein the agent is a combination of at least any two oxysterols selected from the group comprising 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesteroI, 20S-hydroxycholesterol, and 22S-hydroxycholesterol or a portion of any one of 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol active in inducing osteoblastic differentiation.

3. The method of claim 1, wherein the mammalian mesenchymal stem cells are pre-treated with an oxysterol selected from the group comprising 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol or a portion of any one of 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol active in inducing osteoblastic differentiation.

4. The method of claim 1, further comprising treating the mammalian mesenchymal cells with at least one secondary agent selected from the group comprising parathyroid hormone, sodium fluoride, insulin-like growth factor I, insulin-like growth factor II or transforming growth factor beta.

5. The method of claim 1, further comprising treating the mammalian mesenchymal cells with at least one secondary agent selected from the group comprising cytochrome P450 inhibitors, phospholipase activators, arachadonic acid, COX enzyme activators, osteogenic prostanoids or ERK activators.

6. A method of stimulating mammalian cells to express a level of a biological marker of osteoblastic differentiation which is greater than the level of a biological marker in untreated cells, comprising exposing a mammalian cell to a selected dose of at least one agent, wherein the at least one agent is selected from the group comprising 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol or a portion of any one of 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol active in inducing osteoblastic differentiation.

7. The method of claim 6, wherein the agent is a combination of oxysterols selected from the group comprising 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol or a portion of any one of 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol active in inducing osteoblastic differentiation.

8. The method of claim 6 wherein the biological marker is an increase in at least one of alkaline phosphatase activity, calcium incorporation, mineralization, expression of osteocalcin mRNA or Runx2 DNA binding and protein expression.

9. The method of claim 6 wherein the mammalian cells are selected from the group comprising mesenchymal stem cells, osteoprogenitor cells or calvarial organ cultures.

10-14. (canceled)

15. A method of treating a patient exhibiting clinical symptoms of osteoporosis comprising administering at least one agent at a therapeutically effective dose in an effective dosage form at a selected interval to ameliorate the symptoms of the osteoporosis, wherein the at least one oxysterol is selected from the group comprising 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol or a portion of any one of 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol active in inducing osteoblastic differentiation.

16-21. (canceled)

22. An implant for use in the human body, comprising a substrate having a surface, wherein at least the surface of the implant includes at least one agent selected from the group comprising 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol or a portion of any one of 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol active in inducing osteoblastic differentiation in an amount sufficient to induce bone formation in the surrounding bone tissue.

23. The implant of claim 22, wherein the substrate is formed into the shape of a pin, screw, plate, or prosthetic joint.

24. A medicament for use in the treatment of bone disorders comprising a therapeutically effective dosage of at least one oxysterol selected from the group comprising 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol or a portion of any one of 5-cholesten-3beta, 20alpha-diol 3-acetate, 24-hydroxycholesterol, 24(S),25-epoxycholesterol, and 26-hydroxycholesterol, 4 beta-hydroxysterol, 22R-hydroxycholesterol, 20S-hydroxycholesterol, and 22S-hydroxycholesterol active in inducing osteoblastic differentiation.

25. (canceled)

26. The method of claim 4, wherein the at least one secondary agent comprises at least one of bone morphogenic protein, BMP2, BMP 7, and BMP 14.

27. The method of claim 4, wherein the at least one secondary agent comprises a bisphosphonate, an estrogen receptor modulator, calcitonin, vitamin D, and/or calcium.

28-45. (canceled)

46. A method of effecting the cellular hedgehog signaling pathway by using at least one oxysterol or an active portion of an oxysterol molecule to cause a biological effect regulated by the hedgehog signaling pathway, comprising:
contacting cells with at least one oxysterol or an active portion of an oxysterol; and
observing the cells for an indicator of the desired biological effect.

47. The method of claim 46, wherein the desired biological effect is osteogenesis, bone formation or inhibition of adipocyte formation.

48. The method of claim 47 wherein the indicator of the biological effect of osteogenesis is an increase in at least one of alkaline phosphatase activity, calcium incorporation, mineralization, expression of osteocalcin mRNA, or Runx2 DNA binding and protein expression.
